(19) United States
(12) Patent Application Publication
(10) Pub. No.: US 2021/0179676 A1
(43) Pub. Date: Jun. 17, 2021

(54) FUSION PROTEIN AND NUCLEIC ACID MOLECULE FOR EXOGENOUS STIMULANT-DEPENDENT STRESS GRANULE ASSEMBLY

(71) Applicant: ST. JUDE CHILDREN'S RESEARCH HOSPITAL, Memphis, TN (US)

(72) Inventors: J. Paul Taylor, Memphis, TN (US); Peipei Zhang, Memphis, TN (US)

(21) Appl. No.: 16/758,905

(22) PCT Filed: Oct. 26, 2018
(86) PCT No.: PCT/US2018/057651

§ 371 Date: Apr. 24, 2020

Related U.S. Application Data
(63) Continuation-in-part of application No. 15/794,503, filed on Oct. 26, 2017, now Pat. No. 10,526,380.

(51) Int. Cl. C07K 14/415; C12N 9/14

(52) U.S. Cl. CPC C07K 14/415; C12N 9/14; C07K 2319/00; C12Y 306/04013; C12Y 306/04012

(57) ABSTRACT

A nucleic acid molecule encoding a fusion protein composed of an inducible multimerization moiety at the amino terminus, and a GTPase-Activating Protein SH3 Domain-Binding Protein (G3BP) is provided for exogenous stimulus/G3BP-mediated stress granule formation. Further disclosed are chemical or light inducible multimerization proteins or protein domains that can be used for the nucleic acid molecule production, and a method of inducing stress granule formation in a cell comprising expressing the nucleic acid molecule.

### INTRODUCTION

This application is a continuation-in-part of U.S. application Ser. No. 15/794,503, filed Oct. 26, 2017, the content of which is incorporated herein by reference in its entirety.

### BACKGROUND

Stress granules are non-membranous assemblies of mRNA and protein (mRNP) that form when translation initiation is limiting, which occurs during many stress responses including glucose starvation, heat stress, osmotic stress, and oxidative stress. Stress granules are thought to influence mRNA function, localization, and to affect signaling pathways. Normally, stress granule formation is a dynamic, reversible process that relies on particular RNA-binding proteins that harbor self-interacting domains of low sequence complexity (LC domains). However, a disturbance in the assembly and/or dynamics of these structures is closely associated with a wide array of human diseases, including cancer, infectious diseases and neurodegenerative diseases such as Alzheimer's, Huntington's, Parkinson's, frontotemporal dementia (FTD), and amyotrophic lateral sclerosis (ALS).

The GTPase-Activating Protein SH3 Domain-Binding Proteins (G3BPs), G3BP1, G3BP2a and G3BP2b, are important regulators of stress granule dynamics. G3BP1 has been reported to play a critical role in the secondary aggregation step of stress granule formation, and has been used as a reliable marker of stress granules. The misregulation of stress granule dynamics has been reported in many forms of ALS. G3BP1 is critical for neuronal survival since G3BP1 null mice demonstrate widespread neuronal cell death in the central nervous system. Although single knockout of either G3BP1 or G3BP2 partially reduces the number of stress granule-positive cells induced under stress conditions, the knockout of both genes eliminates stress granule assembly.

To facilitate the analysis of G3BP function, G3BP1 has been fused to, e.g., Green Fluorescent Protein (GFP). However, G3BP fusion proteins for selectively inducing stress granule formation have not been described. Rather, conventional approaches of using sodium azide, arsenite, osmotic (e.g., sorbitol), hypoxia, and heat shock are disclosed for stimulating stress granule assembly. Notably, these toxic conditions confound studies for assessing the role of stress granules in diseases such as ALS, FTD, and cancer. Therefore, there is a need in the art for a noninvasive method of inducing stress granule formation in cells.

### SUMMARY OF THE INVENTION

The present invention provides a nucleic acid molecule encoding a fusion protein composed of (a) an inducible multimerization moiety at the amino terminus of the fusion protein, (b) GTPase-Activating Protein SH3 Domain-Binding Protein (G3BP) and a reporter protein. In some embodiments, the inducible multimerization moiety is a chemical or light inducible protein or protein domain such as FK506 Binding Protein (FKBP); plant cryptochrome (CRY, e.g., lacking a Cryptochrome C-terminal Extension (CCE) domain); a light-oxygen-voltage-sensing (LOV) domain; a LOV domain-containing protein; UV-B photoreceptor; N-terminal domain of cryptochrome-interacting basic-helix-loop-helix protein (CIBN); phytochrome interacting factor (PIF); Flavin-binding, Kelch repeat, F-box 1 (FKF1); GIGANTEA, TULIPS, or Dronpa. In certain embodiments, the G3BP lacks an N-terminal Nuclear Transport Factor 2 (NTF2)-like domain. In particular embodiments, the G3BP has the amino acid sequence of SEQ ID NO:25 or SEQ ID NO:28. A vector containing the nucleic acid molecule and cell harboring the vector are also provided, as is a method for inducing stress granule formation in a cell by expressing the nucleic acid molecule in a cell and exposing the cell to an exogenous stimulus (e.g., a chemical or light) that promotes the multimerization of the inducible multimerization domain.

### BRIEF DESCRIPTION OF THE DRAWINGS

FIG. 1A-1B depict an amino acid sequence alignment of human G3BP1 (P1), G3BP2a (P2A) and G3BP2b (P2B) proteins. N-terminal Nuclear Transport Factor 2 (NTF2)-like domains are underlined. Boxes indicate ribonucleoprotein (RNP) motifs RNP1 and RNP2 of the RNA Recognition Motif (RRM). “*” indicate arginine-glycine-rich boxes.

FIG. 2A-2C depict an amino acid sequence alignment of rat (Rattus norvegicus), mouse (Mus musculus), cow (Bos taurus), monkey (Macaca mulatta), human (Homo sapiens), chimp (Pan troglodytes) and dog (Canis lupus) G3BP1 proteins. NTF2-like domains are underlined. “*” indicates identical residues across species. “:” and “.” indicate conserved residues and “-” indicates a gap.

FIG. 3A-3D depict an amino acid sequence alignment of rat (Rattus norvegicus), mouse (Mus musculus), cow (Bos taurus), monkey (Macaca mulatta), human (Homo sapiens), chimp (Pan troglodytes) and dog (Canis lupus) G3BP2a (“A”) and G3BP2b (“B”) proteins. NTF2-like domains are underlined. “*” indicates identical residues across species. “:” and “.” indicate conserved residues and “-” indicates a gap.

FIG. 4A-4F depict an amino acid sequence alignment of cryptochrome (CRY) proteins from plants. OS1 and OS2, Oryza sativa CRY1 and CRY2, respectively; SB1 and SB2, Sorghum bicolor CRY1 and CRY2, respectively; AT1 and AT2, Arabidopsis thaliana CRY1 and CRY2, respectively; LE1, Lycopersicon esculentum CRY1; GM1 and GM2, Glycine max CRY1 and CRY2, respectively; and PP1, Physcomitrella patens CRY1. “*” indicates identical residues across species. “:” and “.” indicate conserved residues and “-” indicates a gap. The characters “F” and “M” above sequences indicate residues known to interact with flavin adenine dinucleotide (FAD) or methenyltetrahydrofolate (MTHF), respectively. “$” indicates trp-triad residues and filled bar indicates the approximate junction between photolyase homology region (PHR) and the Cryptochrome C-terminal Extension (CCE) domains.

### DETAILED DESCRIPTION OF THE INVENTION

It has now been discovered that membrane-less organelle assembly depends upon a very limited number of “nucleator” proteins capable of providing identity and seeding the assembly of a membrane-less organelle. There are very few and perhaps only a single nucleator protein for each organelle. Indeed, it has been discovered that the nucleator protein for stress granules is G3BP1 or G3BP2 and that other stress granule constituent proteins are unable to reconstitute organelle formation. Further, reconstitution of stress granule formation is promoted when the NTF2 domain at the N-terminus is omitted thereby eliminating the negative activity imparted by this domain on the assembly process. In light of this finding, a rapid, uniform and non-toxic approach for induction of stress granules has now been developed. Using the fusion protein and nucleic acids described herein, stress granule formation can be induced in the absence of conventional induction conditions that can confound the analysis of stress granules in disease.

In accordance with this invention, G3BP is fused with an inducible multimerization moiety, e.g. cryptochrome, FKBP1, LOV domain, TULIPS, UVR8 and the like, thereby providing stress granule formation in response to an exogenous stimulus, e.g., light or a chemical. Accordingly, this invention is a fusion protein composed of an inducible multimerization moiety or dimerization moiety (also referred to herein as “IDM”) and G3BP, as well as a method for inducing stress granule formation in a cell by exposing a cell expressing the fusion protein to an exogenous stimulus that induces multimerization of the multimerization moiety.

As is conventional in the art, the term “fusion protein” refers to a protein composed of a plurality of polypeptide components, that while typically unjoined in their native state, are joined by their respective amino and carboxyl termini through a peptide linkage to form a single continuous polypeptide. Fusion proteins may be a combination of two, three or even four or more different proteins. The term fusion protein includes, but is not limited to, a fusion protein with two or three heterologous amino acid sequences; immunologically tagged proteins; and fusion proteins with detectable fusion partners, e.g., reporter proteins such as a fluorescent protein, β-galactosidase, luciferase, and the like. Ideally, a fusion protein comprises or consists essentially of all or a portion of G3BP that is capable of mediating stress granule formation, directly or indirectly linked at its N-terminus to a multimerization moiety. In certain embodiments, the N-terminal NTF2-like domain of G3BP is replaced or substituted with a multimerization moiety; or a multimerization moiety and a reporter protein.

### GTPase-Activating Protein SH3 Domain-Binding Proteins

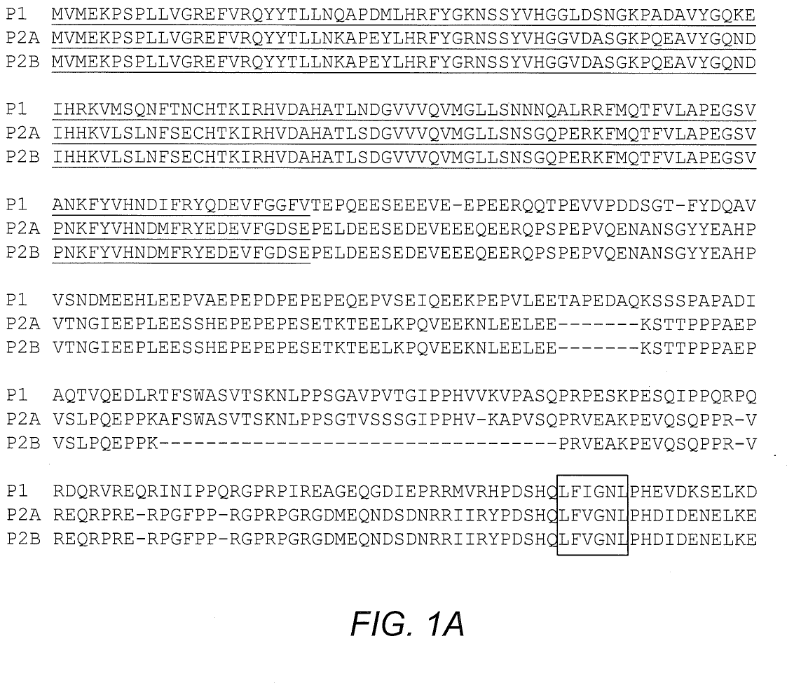

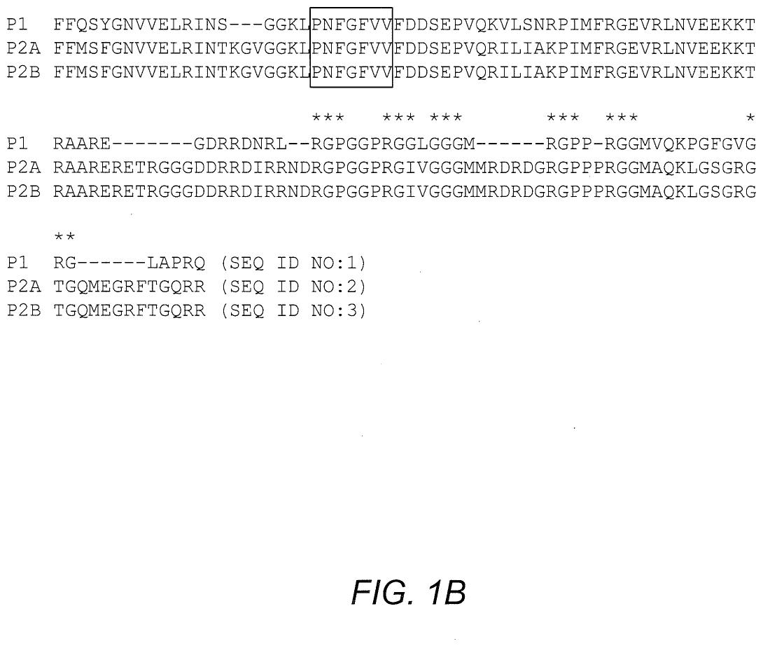

It has been shown that knockout of either G3BP1 or G3BP2 reduces stress granule formation and that knockout of both G3BP1 and G3BP2 eliminates stress granule assembly (Matsuki, et al. (2013) Genes Cells 18(2):135-46). Accordingly, for the purposes of this invention “GTPase-Activating Protein SH3 Domain-Binding Protein” or “G3BP” is intended to include the proteins G3BP1, G3BP2a, and G3BP2b. G3BP2a and G3BP2b are encoded by the same gene and represent alternatively spliced isoforms that differ by an insertion of 99 base pairs in the central region of G3BP2a giving rise to the presence of five SH3-binding domains in G3BP2b compared to four domains in the G3BP2a protein. The amino acid sequence of wild-type human G3BP1 (SEQ ID NO:1) is known in the art and available under GENBANK Accession Nos. NP_005745 and NP_938405 (See FIG. 1A-1B). Likewise, the amino acid sequences of wild-type human G3BP2a (SEQ ID NO:2) and human G3BP2b (SEQ ID NO:3) are known in the art and available under GENBANK Accession Nos. NP_036429 and NP_987100, respectively (See FIG. 1A-1B).

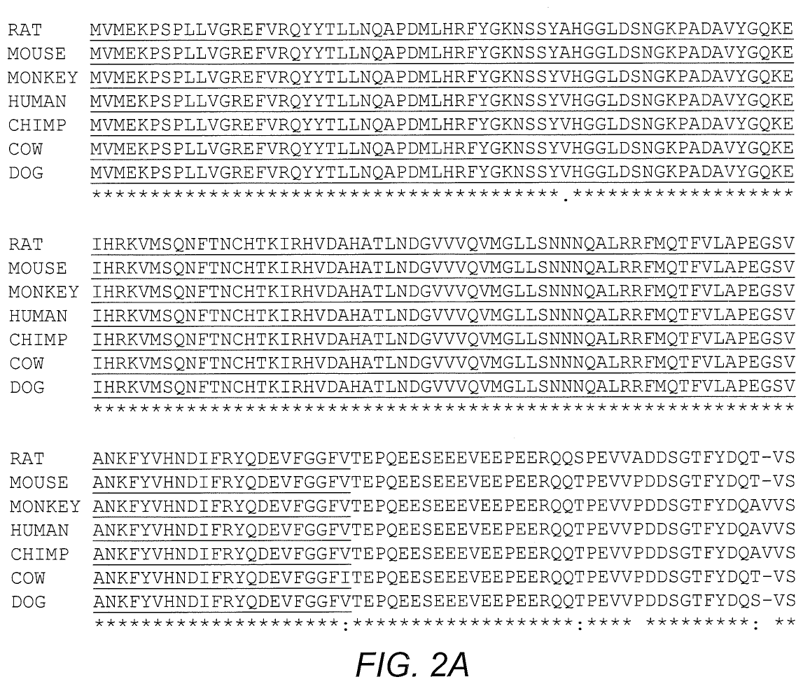

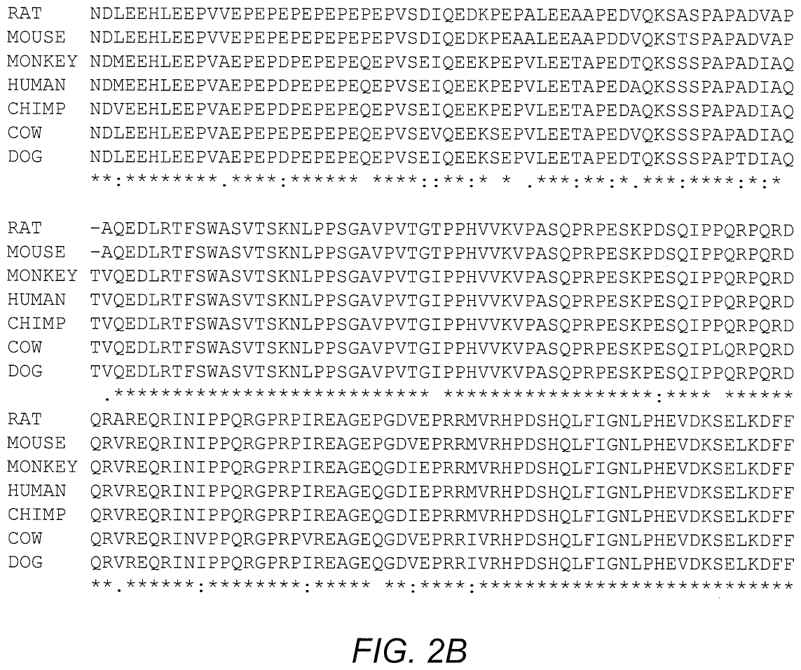

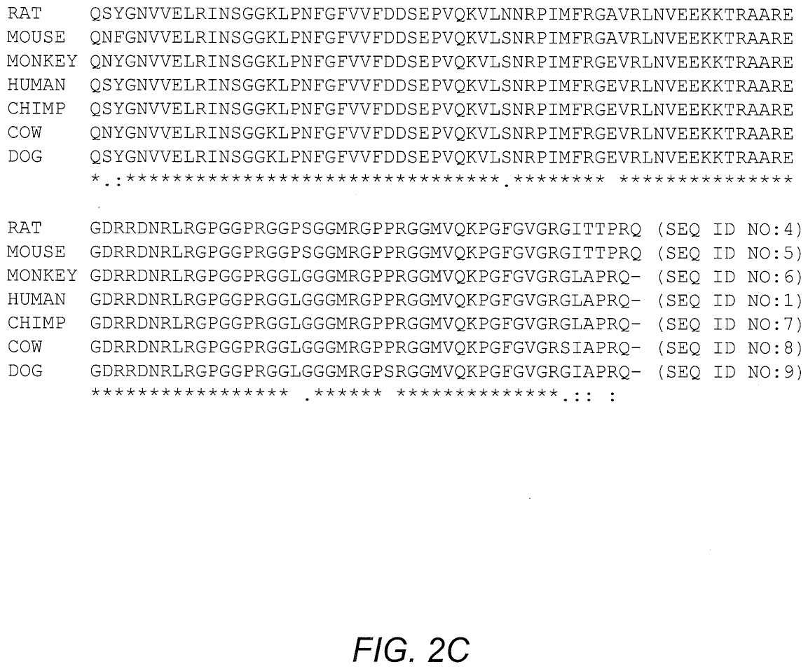

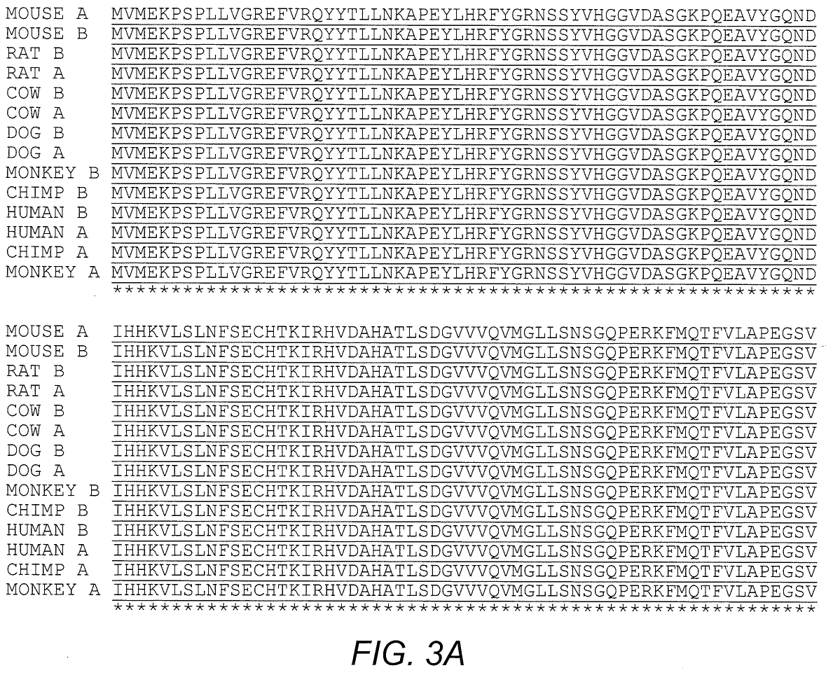

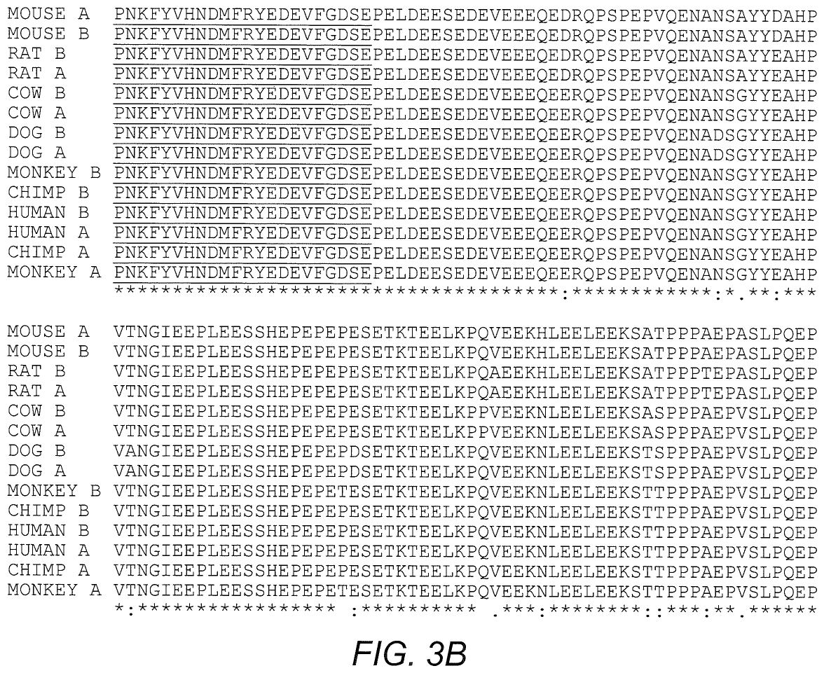

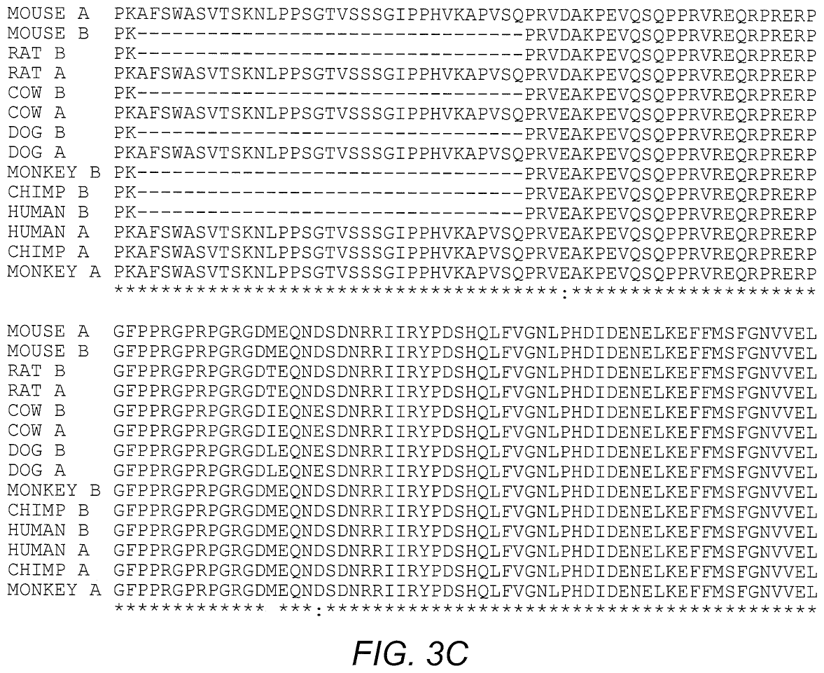

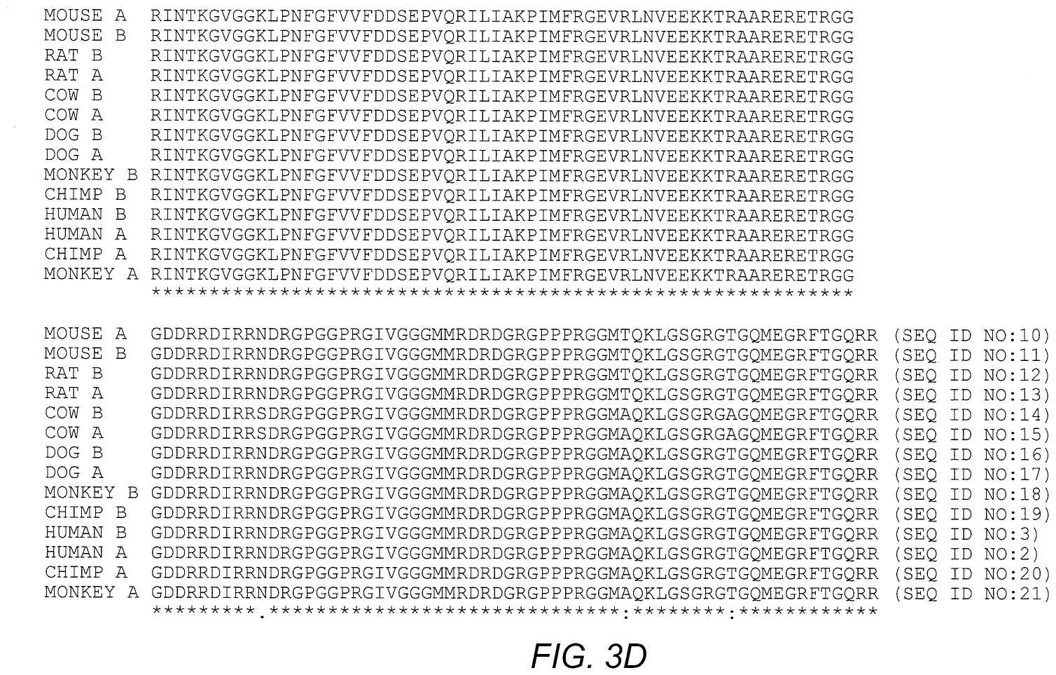

G3BP1, G3BP2a, and G3BP2b are highly conserved across species (see FIG. 2A-2C and FIG. 3A-3D). For example, there is 65% identity and 74% sequence similarity between G3BP1 and G3BP2a proteins in mice and humans. In this respect, this invention also includes the use of both human and non-human G3BP proteins in the fusion protein described herein. In particular, this invention includes G3BP proteins from various animals including chimpanzee, mouse, rat, and the like. Preferably, the animal is a mammal. Examples of wild-type mammalian G3BP proteins are known in the art and available under the GENBANK Accession Nos. provided in Table 1.

TABLE 1

GENBANK Accession No.

| Animal | G3BP1 | G3BP2 |
|---|---|---|
| Pan troglodytes | JAA44555 | JAA39401 |
| | | JAA39402 |
| Macaca mulatta | NP_001248671 | AFE81132 |
| | | NP_001248697 |
| Canis lupus | XP_867372 | XP_022269103 |
| | | XP_022269104 |
| Mus musculus | NP_038744 | NP_001074266 |
| | | NP_001074265 |
| Bos taurus | NP_001032700 | NP_001039920 |
| | | XP_015327172 |
| Rattus norvegicus | NP_598249 | EDL88604 |
| | | NP_001014011 |

Exemplary mammalian G3BP1 and G3BP2 proteins of use in the fusion protein of this invention are presented in FIG. 2A-2C and FIG. 3A-3D, respectively, and include a G3BP1 of SEQ ID NO:1, 4, 5, 6, 7, 8 or 9 or a G3BP2 of SEQ ID NO:2, 3, 10, 11, 12, 13, 14, 15, 16, 17, 18, 19, 20 or 21. In particular embodiments, the fusion protein of the invention includes a human G3BP1 protein of SEQ ID NO:1, or human G3BP2 protein of SEQ ID NO:2, 3 or 22.

Wild-type G3BP proteins feature a highly conserved N-terminal Nuclear Transport Factor 2 (NTF2)-like domain. The NTF2-like domain has been implicated in several G3BP functions including dimerization and stress granule assembly (Tourrière, et al. (2003) J. Cell Biol. 160:823-831). In addition, the G3BP NTF2-like domain has been suggested to play a role in nuclear shuttling. This suggestion is based on findings of G3BP1 and G3BP2 both in the cytoplasm and in the nucleus (Barnes, et al. (2002) Cancer Res. 62:1251-1255; French, et al. (2002) Histochem. J. 34:223-231). Also, NTF2-like domain deletion mutants of G3BP2a have been shown to be exclusively localized to the cytoplasm (Prigent, et al. (2000) J. Biol. Chem. 275:36441-36449). In accordance with certain embodiments of this invention, the NTF2-like domain of G3BP is absent in the instant fusion protein. Accordingly, “G3BP lacking an NTF2-like domain” refers to the deletion or removal of the NTF2-like domain of G3BP. As is known in the art, the NTF2-like domain of G3BP is located within the N-terminal ˜140 amino acid residues of G3BP (see FIG. 1A-1B). Accordingly, “G3BP lacking an NTF2-like domain” refers to deletion of, e.g., residues 1-139, 7-135, 11-134, 1-142, 7-142, 11-142 or 11-139 of a wild-type G3BP1, G3BP2a or G3BP2b protein.

G3BP C-termini have two motifs traditionally associated with RNA binding. These include a canonical RNA Recognition Motif (RRM) and loosely conserved RGG (arginine-glycine rich) boxes. The RRM domain is composed of two short, loosely conserved motifs, RNP1 (LFIGNL; SEQ ID NO:23) and RNP2 (PNFGFVVF; SEQ ID NO:24), separated by 30 to 33 amino acid residues and has been shown to bind to RNA molecules (U.S. Pat. No. 8,268,550; Pin, et al. (2017) Acta Veterinaria et Zootechnica Sinica 48(3):515-521). RGG domains (RGP, RGG, GGG and GRG) located at the C-terminus of G3BP are often found in RNA-binding proteins and may confer cooperative binding to RRM motifs. Therefore, in accordance with the fusion protein of this invention, a “G3BP lacking an NTF2-like domain” refers to a G3BP having an RNA Recognition Motif comprising the amino acid sequence of SEQ ID NO:23 and SEQ ID NO:24, and five or six arginine-glycine rich boxes. An exemplary human G3BP1 protein lacking an NTF2-like domain, which is of particular use in the fusion protein of this invention is provided under SEQ ID NO:25. Exemplary human G3BP2 proteins lacking an NTF2-like domain, which are of particular use in the fusion protein of this invention are provided under SEQ ID NOs:26, 27 and 28. Exemplary non-human mammalian G3BP1 proteins lacking an NTF2-like domain are provided under SEQ ID NOs:29, 30, 31, 32, 33, 34 and 35. Exemplary non-human mammalian G3BP2 proteins lacking an NTF2-like domain are provided under SEQ ID NOs:35, 36, 37, 38, 39, 40, 41, 42, 43, 44, 45 and 46. In particular embodiments, the fusion protein of the invention includes a G3BP1 protein of SEQ ID NO:25.

Notably, it has been shown that G3BP1 lacking the N-terminal NTF2-like domain does not induce stress granule formation (Takahashi, et al. (2013) Mol. Cell Biol. 33:815-829; Tourriere, et al. (2003) J. Cell Biol. 160:823-31). However, as described herein, a fusion protein including (a) a cryptochrome at the amino terminus and (b) a G3BP lacking an NTF2-like domain at the carboxy terminus restores stress granule formation and imparts light-sensitivity to G3BP. Similarly, fusion of a cryptochrome to a full length G3BP retains stress granule formation and imparts light-sensitivity to G3BP. Accordingly, the fusion protein of this invention includes a cryptochrome, in particular a plant cryptochrome, for providing light-sensitive G3BP-mediated stress granule formation.

### Inducible Multimerization Moieties

The term “multimerization” refers to the association of two or more moieties into a macromolecular complex via a non-covalent interaction. In some embodiments, the multimer is formed by two proteins. In certain embodiments, the multimerization moiety is a dimerization moiety. In other embodiments, the multimer is formed by, e.g., two magnets. In some embodiments, the multimer formed is a proteinaceous homodimer (i.e., referred to homodimerization), wherein the two proteins of the dimer are substantially the same protein. In accordance with this embodiment, only a single fusion needs to be produced and expressed for a dimer to form. In other embodiments, the dimer formed is a proteinaceous heterodimer (i.e., referred to heterodimerization), wherein the two proteins of the dimer are substantially different. In accordance with this embodiment, two fusion proteins are produced, each containing a protein of the heterodimer fused to G3BP1. Ideally, the homodimer or heterodimer brings two or more G3BP1 proteins in close enough proximity so serve as a nucleation site for assembly of a stress granule.

In accordance with the present invention, the association of at least two multimerization moieties is brought about by the introduction of an exogenous stimulus. The exogenous stimulus can be an environmental signal, e.g., light, heat, sound, pressure, magnetic field, or electrical current; a chemical signal such as a small molecule or ligand (e.g., chemical inducible dimerization (CID) system), or a combination thereof. In accordance with the present fusion protein, the inducible multimerization moiety includes one or more domains that bind/recognize the stimulus (e.g., a ligand, chemical or chromophore) and mediate multimerization (i.e., self-dimerization or heterodimerization).

The most widely used CID systems are based on FK506 Binding Protein (FKBP), which binds tightly to the immunosuppressant drugs FK506 and rapamycin. One example of such a homodimerization system uses a synthetic derivative of FK506 with two FKBP-binding moieties (i.e., FK1012; Spencer, et al. (1993) Science 262:1019-24), which induces the homodimerization of FKBP-fusion proteins. A heterodimerization system exploits the ligand-mediated interaction between FKBP and mTOR (Rivera, et al. (1996) Nat. Med. 2:1028-32). An 89-amino acid fragment from mTOR called FRB is sufficient for binding the FKBP-rapamycin complex but does not bind FKBP in the absence of rapamycin. Rapamycin addition induces the heterodimerization of FRB- and FKBP-fused proteins rapidly, on the order of seconds, yet irreversibly due to the high affinity of the FKBP-FRB interaction. To ensure that the rapamycin only binds to the engineered FRB and not to endogenous mTOR, several rapamycin analogues such as iRap, AP21967, and AP23102 have been developed (Inoue, et al. (2005) Nat. Meth. 2:415-8; Putyrski & Schultz (2012) FEBS Lett. 586:2097-105).

Like FK506 and rapamycin, plant hormones have also been used as multmizers. For example, abscisic acid (ABA) has been used to induce the dimerization of PYR1-like (PYL) and ABA insensitive 1 (ABI1) proteins (Liang, et al. (2011) Sci Signal. 4(164):rs2). Similarly, gibberellic acid (GA₃) and the gibberellin analog GA₃-AM have been shown to rapidly induce the dimerization of gibberellin insensitive (GAI) and gibberellin insensitive dwarf1 (GID1) proteins (Miyamoto, et al. (2012) Nat. Chem. Biol. 8:465-70). The GAI-GID1 system, like FKBP-FRB, functions on the order of seconds but does not reverse upon washout.

In addition to multimerization systems based on plant hormones, other synthetic heterodimerizers have been created by covalently linking two orthogonal ligands, enzyme substrates, or protein-targeting tags (Rutkowska & Schultz (2012) Angew Chem. Int. Ed. Engl. 51:8166-76). For example, G-protein-coupled receptors (GPCRs) activated solely by synthetic ligands (RASSLs) have been developed (Coward, et al. (1998) Proc. Natl. Acad. Sci. USA 95:352-7; Conklin, et al. (2008) Nat. Methods 5:673-8). These receptors are designed to be insensitive to endogenous ligands but responsive to exogenously added synthetic ligands. First-generation RASSLs have been used to acutely activate GPCR signaling in cardiac tissue in vivo. However, the synthetic ligands used in these first-generation RASSLs also had high affinity for endogenous GPCRs. A second-generation RASSL technology, known as Designer Receptors Exclusively Activated by Designer Drugs (DREADDs), was therefore developed. In this approach, GPCRs are activated by clozapine-N-oxide, a pharmacologically inert yet bioavailable synthetic ligand (Armbruster, et al. (2007) Proc. Natl. Acad. Sci. USA 104:5163-8). The DREADD methodology has been applied to a wide array of G-protein signaling pathways, including Gq, Gi, Gs, and β-arrestin signaling

For precise spatiotemporal control of multimerization, an optical or light inducible protein (also referred to herein as a “LIP”) can be used in the fusion protein of this invention. In accordance with some embodiments, photoactivatable metal ions, amino acids, second messengers, ligands, or photocaged versions of chemical dimerizers, such as photocaged analogues of rapamycin (Karginov, et al. (2011) J. Am. Chem. Soc. 133:420-3), ABA (Wright, et al. (2015) Chembiochem. 16:254-61) and GA₃ (Schelkle, et al. (2015) Angew Chem. Int. Ed. Engl. 54:2825-9), can be used to combine the aforementioned CID systems with light activation.

In addition to small molecule photoswitches, a photosensitive protein domain light inducible protein can be used as an inducible multimerization moiety in accordance with this invention. Ideally, many of the known light inducible proteins require chromophore cofactors (e.g., FMN or FAD) that are normally present in cells or can be introduced into cells either by incubation in solution (Levskaya, et al. (2009) Nature 461:997-1001) or introduction of the required biosynthetic genes (Muller & Weber (2013) Chem. Commun. (Camb) 49:8970-8972). Exemplary light inducible proteins of use in the fusion protein of this invention include, but are not limited to, CRY (cryptochrome), LOV (light-oxygen-voltage-sensing) domains or proteins containing the same, UVR8 (UV-B photoreceptor), CIBN (N-terminal domain of CIB1 (cryptochrome-interacting basic-helix-loop-helix protein 1)), PIF (phytochrome interacting factor), FKF1 (Flavin-binding, Kelch repeat, F-box 1), GIGANTEA, TULIPS, Dronpa, or combinations thereof.

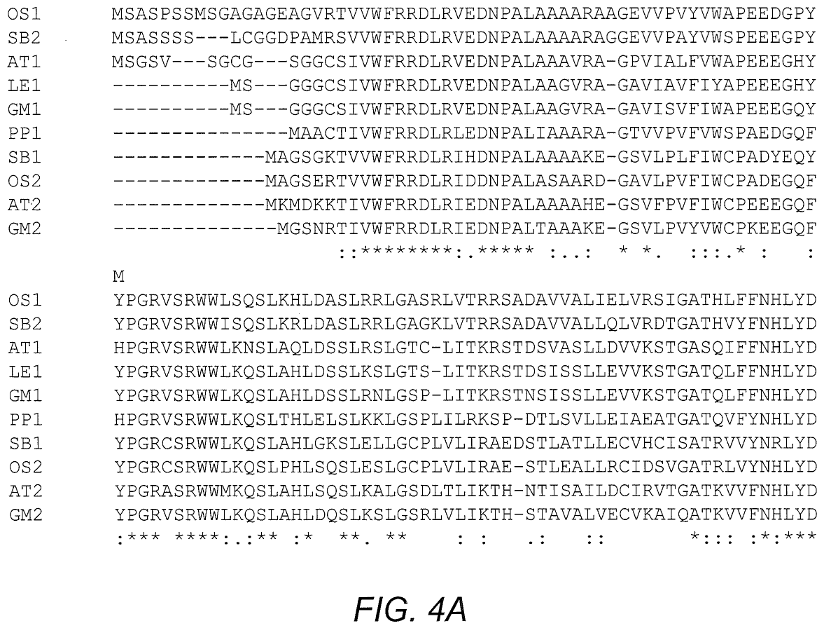

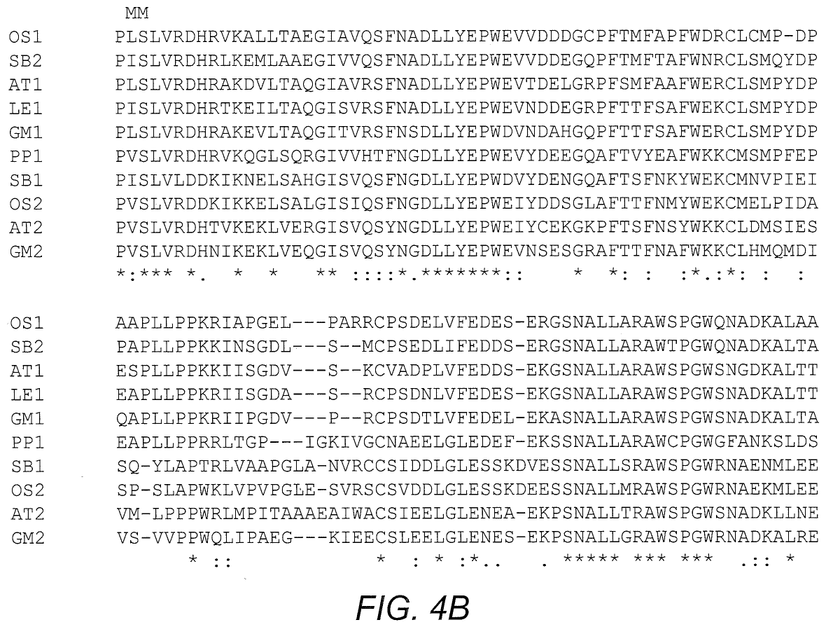

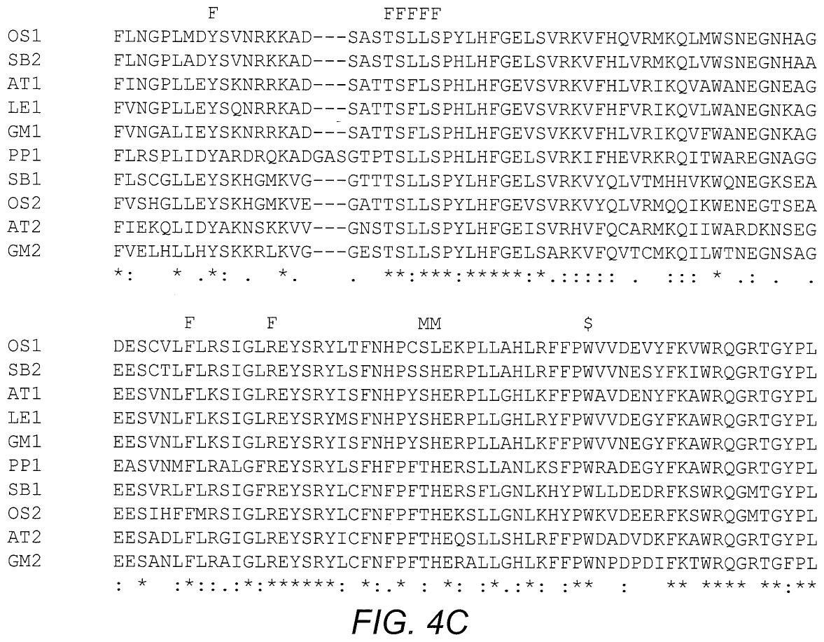

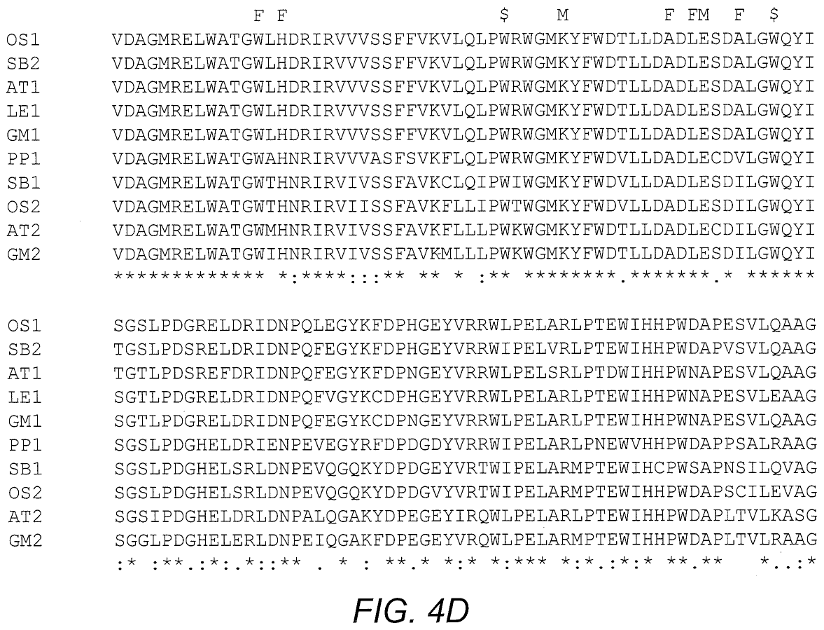

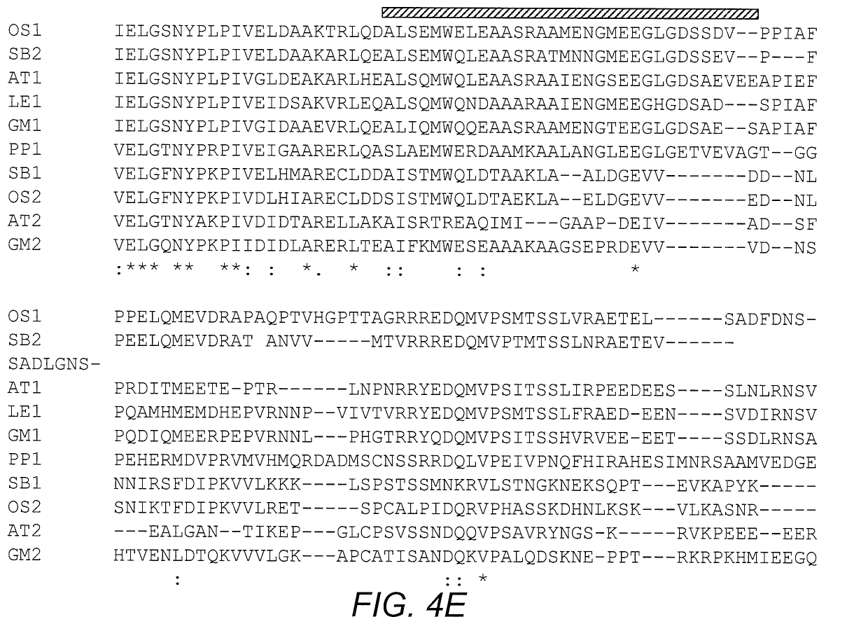

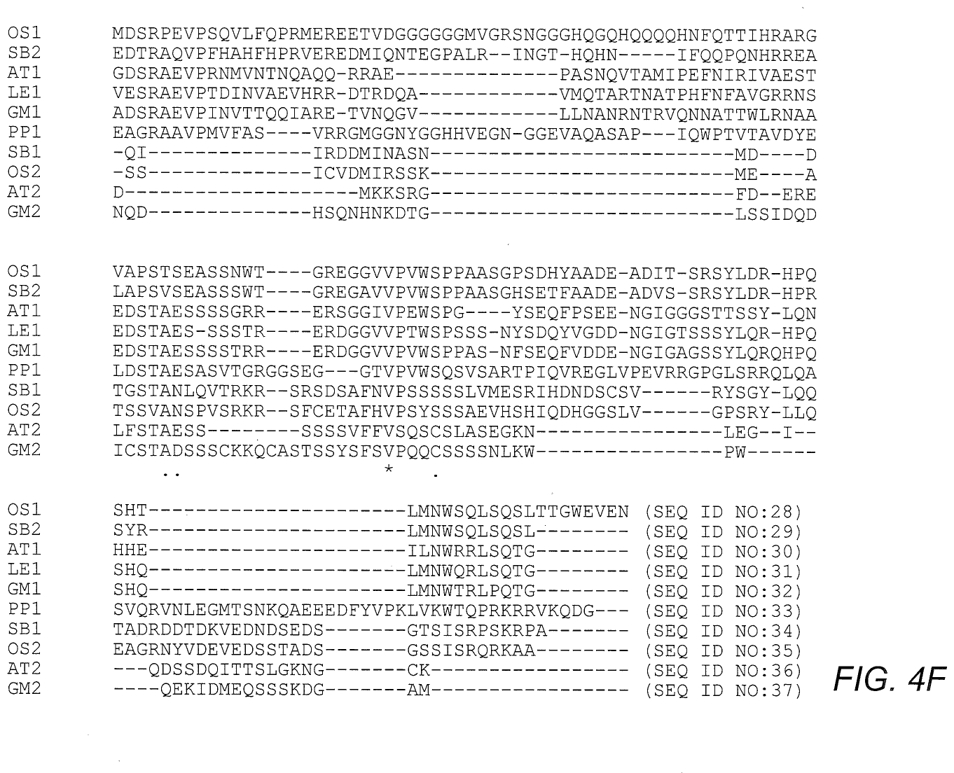

“Cryptochrome” or “CRY” is an ultraviolet-A/blue light photoreceptor found in plants, insects, fish, amphibians, mammals and fungi. Cryptochromes are composed of two major domains, the N-terminal PHR (for Photolyase-Homologous Region) domain of about 500 residues, and the C-terminal extension CCE (for Cryptochrome C-terminal Extension) domain, which varies in length (FIG. 4A-4F). The PHR domain is required for chromophore-binding and homodimerization (Sang, et al. (2005) Plant Cell 17:1569-84; Yu, et al. (2007) Proc. Natl. Acad. Sci. USA 104:7289-94), whereas CCE is an effector domain of cryptochrome (Yang, et al. (2000) Cell 103:815-827; Wang, et al. (2001) Science 294:154-158).

For the purposes of this invention, “cryptochrome” or “CRY” is intended to include the proteins CRY1, CRY2 and CRY3. While CRY proteins from fungi, insects or animals can be used in the fusion protein of this invention, preferably the CRY protein is a plant CRY protein. Plant CRY proteins include, but are not limited to, CRY1 and CRY2 proteins from Chlamydomonas reinhardtii, Physcomitrella patens, Adiantum capillus-veneris, Arabidopsis thaliana, Lycopersicon esculentum, Sorghum bicolor, Oryza sativa, Glycine max and Sinapis alba (Lin & Todo (2005) Genome Biology 6:220) (Table 2).

TABLE 2

GENBANK Accession No.

| Plant | CRY1 | CRY2 |
|---|---|---|
| Physcomitrella patens | XP_001751763 | — |
| Arabidopsis thaliana | NP_567341 | NP_171935 |
| Lycopersicon esculentum | NP_001234667 | — |
| Sorghum bicolor | XP_002436988 | AAV97867 |
| Oryza sativa | BAD17529 | BAD23780 |
| Glycine max | NP_001242152 | NP_001235220 |

CRY1 = DSPD, PHLL1; CRY2 = KIAA0658, PHLL2.

The CRY PHR domain is composed of sequential α/β subdomains and α-helix subdomains, large parts of which cover the chromophore binding sites of 5,10-methenyltetrahydrofolate (MTHF) and flavin adenine dinucleotide (FAD). In addition to the roles of binding chromophores to perceive light and get photoactivated, the PHR domain mediates self-dimerization and blue light-induced autophosphorylation, both of which are essential for CRY activity. The FAD-binding pocket of cryptochrome is the most conserved region within the PHR domain (see FIG. 4C-4D). In addition, W324, W377, and W400 of the trp-triad residues, which are required for photoreduction, are also conserved (see FIG. 4C-4D). Accordingly, in certain embodiments, the CRY used in the fusion protein of this invention includes a PHR domain required for binding chromophores, self-dimerization and blue light-induced autophosphorylation.

Although the CCE domains of plant cryptochromes share little sequence similarity with the CCE domains of animal cryptochromes, plant cryptochromes from different species do share a common sequence DAS motif in their CCE's (Lin & Shalitin (2003) Annu. Rev. Plant Biol. 54:81469-496). Cryptochromes from liverwort, moss, and fern all possess various versions of the DAS motif (Lin & Shalitin, 2003). Computational analyses of secondary structures of CCEs from Arabidopsis and human cryptochromes predict that this domain is intrinsically unstructured. The unstructured nature of the CCE domain of Arabidopsis CRY1 (the C-terminal 180 residues; see FIG. 4E-4F) has been confirmed by the circular dichroism and NMR analyses. It has been suggested that the CCE domains of cryptochromes act as effector modules by undergoing light-induced folding or unfolding to alter their interaction with the PHR domain and to change the overall conformation of the photoreceptors.

It has now been found that a CRY protein lacking a CCE domain is sufficient to facilitate light-dependent, G3BP-mediated stress granule formation. Therefore, the CRY protein used in the fusion protein of this invention may be a full-length CRY protein (e.g., SEQ ID NO:47, 48, 49, 50, 51, 52, 53, 54, 55 or 56) or more particularly a truncated CRY protein lacking a CCE domain. In particular embodiments, the CRY of the fusion protein of this invention comprises, consists essentially of, or consists of the N-terminal PHR domain of a CRY protein. In other embodiments, the CRY protein is an Arabidopsis CRY2 protein with an E490G mutation. Exemplary CRY proteins lacking a CCE domain are provided in SEQ ID NO:57, 58, 59, 60, 61, 62, 63, 64, 65, 66 and 72. In particular embodiments, the fusion protein of the invention includes a CRY2 protein of SEQ ID NO:59, SEQ ID NO:65 or SEQ ID NO:72. In certain embodiments, the fusion protein of this invention has the amino acid sequence set forth in SEQ ID NO:67 or SEQ ID NO:73.

The CRY protein can be used alone or in combination with the N-terminus of CIB1 (i.e., CIBN) to form a heterodimer (Liu, et al. (2008) Science 322(5907):1535-9). An exemplary CIB1 protein is available under GENBANK Accession No. NM_119618 (A. thaliana CIB1).

The phytochromes (PHY) include a family of biliprotein photoreceptors that enable plants to adapt to their prevailing light environment. Phytochromes possess the ability to efficiently photointerconvert between red light (λₘₐₓ, 665 nm) absorbing Pr and far red light (λₘₐₓ, 730 nm) absorbing Pfr forms, a property conferred by covalent association of a linear tetrapyrrole (bilin or phytobilin) with a large apoprotein. Phytochromes from cyanobacteria, to green algae and higher plants are composed of a well conserved N-terminal domain, roughly 390-600 amino acids in length (see, e.g., U.S. Pat. No. 6,046,014), to which the phytobilin prosthetic group is bound. An exemplary phytochrome sequence is disclosed in US 2003/0082809. In some embodiments, the phytobilin of the PHY domain is a linear tetrapyrrole, four pyrroles linked together in a linear molecule with then varying substituents. Preferably, the linear tetrapyrrole is a linear tetrapyrrole occurring in nature, e.g., a linear tetrapyrrole selected from phycocyanonbilin, phycoerythrobilin, phycourobilin, phycoviolobilin, phytochromobilin, biliverdin, bilirubin, mesobiliverdin, mesobilirubin, bilane, bilin, urobilin, stercobilin, and urobilinogen.

In some embodiments the PHY can be used alone or in combination with a PIF (phytochrome interacting factor) to form a heterodimer (WO 2013/133643; Kim, et al. (2014) Chem. Biol. 21:903-912).

“UVR8” is a seven-bladed β-propeller protein of 440 amino acid residues in length (Christie, et al. (2012) Science 335:1492-1496; Wu, et al. (2012) Nature 484:214-219). Molecular and biochemical studies have demonstrated that in light conditions devoid of UV-B, the UVR8 photoreceptor exists as a homodimer, which undergoes instant monomerization following UV-B exposure, a process dependent on an intrinsic tryptophan residue that serves as an UV-B chromophore (Rizzini, et al. (2011) Science 332:103-106). Accordingly, in accordance with embodiment of the invention, multimerization is induced in the absence of UV-B light. Alternatively, when used in combination with COP1, a light-induced UVR8-COP1 heterodimer can be formed (Rizzini, et al. (2011) Science 332:103-106; Crefcoeur, et al. (2013) Nat. Commun. 4:1779).

A eukaryotic, blue light-regulated gene system based on the interaction of FKF1 (flavin-binding, kelch repeat, F-box 1) and GI (GIGANTEA) from Arabidopsis thaliana has also be developed.

The “light-oxygen-voltage-sensing” or “LOV” domains superfamily are a group of light-sensing domains that bind flavins as prosthetic groups and act as reversible photoswitches in bacteria, fungi and plants. LOV-domain-containing photoreceptors control functionally heterogeneous effector domains such as serine/threonine kinases, e.g., in the flowering plant Arabidopsis thaliana (Kinoshita, et al. (2001) Nature 414:656-660) or the green alga Chlamydomonas reinhardtii (Huang, et al. (2002) Physiol Plant. 115:613-622); or transcriptional regulators, e.g., in the fungus Neurospora crassa (Heintzen & Liu (2001) Adv. Genet. 58:25-66) or in the yellow-green alga Vaucheria frigida (Takahashi, et al. (2007) Proc. Natl. Acad. Sci. USA 104(49):19625-30). When exposed to blue light (440-473 nm) LOV domains undergo a conformational change, leading to allosteric control of effector domains. An exemplary LOV domain includes residues 180 to 312 of Ochromonas danica aureochrome1 like protein (Uniprot Accession No. C5NSW6). Another LOV domain of use in the invention is located in the C-terminus of a ureochrome1 of V. frigida (Takahashi, et al. (2007) Proc. Natl. Acad. Sci. USA 104(49):19625-30).

The tunable light-controlled interacting protein tags (TULIPs) make use of a blue light-sensing LOV domain and an engineered PDZ domain. Specifically, the LOV2 domain of Avena sativa phototropin 1 (AsLOV2) and an engineered PDZ domain (ePDZ) are expressed and can recruit proteins fused thereto to various locations in the cells, either globally or with precise spatial control using a steerable laser. The equilibrium binding and kinetic parameters of the interaction are tunable by mutation, making TULIPs readily adaptable to signaling pathways with varying sensitivities and response times. See Strickland, et al. (2012) Nat. Methods 9(4):379-384.

As a homologue of Aequorea GFP, Dronpa autogenically forms a visible wavelength chromophore within the protected interior of its β-barrel structure. Dronpa rapidly converts between a dark state and a bright state upon illumination with 490 nm and 400 nm light, respectively. Therefore, Dronpa mutants that either dimerize or tetramerize in the bright state but remain monomeric in the dark state have been generated and fused to proteins such as a guanine nucleotide exchange factor (GEF) or protease (Zhou, et al. (2013) Science 338(6108):810-4). When in the bright state, the two Dronpa domains form an interface and upon exposure to 400 nm light, the interface breaks.

Functionalized magnetic nanoparticles can also be used to control stress granule formation. For example, it has been shown that forces generated by applying a magnetic field on a nanoparticle can be used to manipulate mechanotransduction in living cells (Hughes, et al. (2008) J. R. Soc. Interface 5:855-63). Furthermore, ligand-conjugated magnetic nanoparticles have been used to activate receptors, such as FCεRI and EGFR, by mediating their clustering in the membrane (Mannix, et al. (2008) Nat. Nanotechnol. 3:36-40; Bharde, et al. (2013) PLoS One 8:e68879).

In some embodiments, the fusion protein of this invention also includes a reporter protein. As is conventional in the art, a reporter protein is a protein that can allow for the detection, quantification, localization and/or isolation of a protein of interest. Ideally, a reporter protein of use in this invention is a fluorescent protein or a combination of fluorescent proteins. The fluorescent protein can be or include an ultraviolet fluorescent protein, a blue fluorescent protein, a cyan fluorescent protein, a green fluorescent protein, a yellow fluorescent protein, an orange fluorescent protein, a red fluorescent protein, a far-red fluorescent protein, a near infrared fluorescent protein, an infrared fluorescent protein, a sapphire-type fluorescent protein, a long Stokes shift fluorescent protein, a switchable fluorescent protein, or any combination thereof. In some embodiments, the fluorescent protein has an excitation wavelength that overlaps with the response range of light-inducible protein of the instant fusion protein. In other embodiments, the fluorescent protein has an excitation wavelength that does not overlap with the response range of the light-inducible protein of the instant fusion protein. Notably, light-inducible proteins such as CRYs are active principally in the range of 365 to 550 nm, with a maximal response in the range of 390 to 480 nm. Examples of suitable fluorescent proteins are provided in Table 3.

TABLE 3

| Fluorescent Protein | Excitation max (nm) | Emission max (nm) |
|---|---|---|
| Blue Fluorescent Proteins | | |
| Azurite | 384 | 450 |
| EBFP | 383 | 445 |
| EBFP2 | 383 | 448 |
| Y66H | 382 | 459 |
| Cyan Fluorescent Proteins | | |
| ECFP | 439 | 476 |
| AmCyan1 | 458 | 489 |
| Cerulean | 433 | 475 |
| CyPet | 435 | 477 |
| mTFP1 | 462 | 492 |
| TagCFP | 458 | 480 |
| Green Fluorescent Proteins | | |
| AcGFP | 480 | 505 |
| Azami Green | 492 | 505 |
| Emerald | 487 | 509 |
| GFP | 395 | 509 |
| Sterner | 395 | 509 |
| TagGFP | 482 | 505 |
| T-Sapphire | 399 | 511 |
| TurboGFP | 482 | 502 |
| ZsGreen | 493 | 505 |
| Yellow Fluorescent Proteins | | |
| EYFP | 514 | 527 |
| mBanana | 540 | 553 |
| mCitrine | 516 | 529 |
| TagYFP | 508 | 524 |
| Topaz | 514 | 527 |
| Venus | 515 | 528 |
| YPet | 517 | 530 |
| Orange Fluorescent Proteins | | |
| RFP | 558 | 583 |
| Tomato | 554 | 581 |
| Kusbira Orange | 548 | 559 |
| mOrange | 548 | 562 |
| mTangerine | 568 | 585 |
| Red Fluorescent Proteins | | |
| AsRed2 | 576 | 592 |
| HcRedl | 588 | 618 |
| JRed | 584 | 610 |
| mApple | 568 | 592 |
| mCherry | 587 | 610 |
| mPlum | 590 | 649 |
| mRaspberry | 598 | 625 |
| mRFP1 | 584 | 607 |
| mRuby | 558 | 605 |
| mStrawberry | 574 | 596 |

Reporter proteins other than fluorescent reporter proteins can be employed in addition to or in the alternative to fluorescent reporter proteins. For example, antibodies, antibody fragments, peptide tags (e.g., His6×, FLAG), enzymes, or the like, or any combination thereof can be used. The reporter protein can be fused (in-frame) to the N-terminus (e.g., Reporter-IDM-G3BP) or C-terminus (e.g., IDM-G3BP-Reporter) of the fusion protein or be inserted between the IDM and G3BP proteins (e.g., IDM-Reporter-G3BP). Exemplary CRY-G3BP fusion proteins including a reporter protein are set forth in SEQ ID NO:68, SEQ ID NO:70 and SEQ ID NO:74.

The fusion protein of this invention can be prepared by conventional recombinant DNA methods. In general, this includes isolating the nucleic acid molecule encoding the G3BP and IDM proteins of interest (e.g., by restriction enzyme digestion or PCR amplification); inserting the coding sequence of G3BP and IDM (in frame) into a suitable vector, e.g., an expression vector that includes the requisite sequences for protein expression (e.g., promoter, terminator, etc.); and introducing the vector into a suitable host cell, e.g., to express the fusion protein. In certain embodiments, this invention provides a nucleic acid molecule encoding an IDM-G3BP fusion protein, a vector including said nucleic acid molecule and a host cell harboring said vector.

The terms “nucleic acid molecule” and “polynucleotide” are used interchangeably and refer to a polymeric form of nucleotides of any length, either deoxyribonucleotides or ribonucleotides, or analogs thereof. Non-limiting examples of nucleic acid molecules include a gene, a gene fragment, exons, introns, messenger RNA (mRNA), transfer RNA, ribosomal RNA, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

In particular, the nucleic acid molecule of the invention encodes the fusion protein disclosed herein. A “coding sequence” or a sequence that “encodes” a selected polypeptide, is a nucleic acid molecule which can be transcribed (in the case of DNA) and translated (in the case of mRNA) into a polypeptide, for example, in a host cell when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from mRNA, genomic DNA sequences, and synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence. Exemplary coding sequences for CRY-G3BP fusion proteins are set forth herein in SEQ ID NO:69 and 71.

To facilitate amplification and expression, the nucleic acid molecule encoding the fusion protein disclosed herein may be inserted into a vector. A “vector” is capable of transferring gene sequences to a host cell. Typically, “vector,” “expression vector,” and “gene transfer vector,” mean any nucleic acid construct capable of directing the expression of a gene of interest and which can transfer gene sequences to host cells, which can be accomplished by genomic integration of all or a portion of the vector, or transient or inheritable maintenance of the vector as an extrachromosomal element. Thus, the term includes cloning, and expression vehicles, as well as integrating vectors.

A number of expression vectors for the expression of a nucleic acid molecule encoding a fusion protein of the invention are known in the art. Different examples of expression vectors are available for expression of the fusion protein in mammalian cells, insect cells, yeast cells, and bacterial cells. For example, the pEGFP-Cl mammalian vector (Invitrogen) contains a CMV promoter sequence, a nucleic acid sequence encoding green fluorescence protein, a multiple cloning site for insertion of nucleic acid sequence encoding the fusion protein. Additional non-limiting examples of publicly-available mammalian expression vectors include constitutive expression vectors GATEWAY® pDEST™26, pDEST™27, pDEST™40, and pDEST™47 (Invitrogen); adenoviral expression vectors (e.g., pAd/CM/V5-Dest GATEWAY® Vector Kit (Invitrogen); episomal expression vectors pCEP4 and pEBNA DEST (Invitrogen); lentiviral expression vectors (e.g., VIRAPOWER™ Bsd; Invitrogen); and regulated expression vectors GATEWAY® pT-REX™-DEST 30 and pT-REX™-DEST 31 (Invitrogen). Non-limiting examples of bacterial expression vectors include GATEWAY® vectors pDEST™14, pDEST™15, pDEST™17, pDEST™24, pET-DEST42; pEM7/Bsd; pEM7/Zeo; pRSET A, B, & C; pRSET-BFP; pRSET-CFP; pRSET-EmGFP; pTrcHis A, B, & C; and pTrcHis2 A, B, & C vectors (Invitrogen). Non-limiting examples of yeast expression vectors include pAO815; pGAPZ A, B, & C; pPIC3.5K; pPIC9K; pTEFl/Bsd; pTEFl/Zeo; pYC2/CT; pYES2; pYES2/CT; and pYES3/CT (Invitrogen). Non-limiting examples of insect and baculovirus expression vectors include GATEWAY® vectors pDEST™10, pDEST20, pDEST™8, pMT-DEST™48; pAC5.1/V5-His A, B, & C; pFastBac Dual; and pIB/V5-His-DEST (Invitrogen).

The expression vectors used to express a fusion protein may include one or more (e.g., 1, 2 or 3) constitutive promoter sequences and/or one or more (e.g., 1, 2 or 3) inducible promoter sequences. Non-limiting examples of constitutive promoter sequences include bacterial promoters (e.g., E. coli a⁷⁰, σ^{s}, σ³², or σ⁵⁴ promoters; B. subtilis σ^{Λ} or σ^{B} promoters; T7 RNA polymerase-based promoters; and a bacteriophage SP6 promoter), yeast promoters (e.g., pCyc, pAdh, pSte5, ADHl, cyc70, cyc43, cyc28, pPGKl, pCYC, and GPD (TDH3) promoters), and mammalian promoters (e.g., cytomegalovirus immediate early gene-based promoters, SV40 early promoter, and Rous sarcoma virus promoter). Non-limiting examples of inducible promoter sequences include alcohol dehydrogenase I gene promoters, tetracycline-responsive promoter systems, glucocorticoid receptor promoters, estrogen receptor promoter, ecdysone receptor promoters, metallothionein-based promoters, and T7-polymerase based promoters. Several different mammalian expression vectors available that allow for the inducible expression of a nucleic acid sequence (e.g., a fusion protein) are publicly available including pTET-ON Advanced (Clontech), pERV3 (Stratagene), pNEBR-Rl (New England BioLabs), and pCMV5-CymR (Qbiogene).

One or more nucleic acid molecules encoding a fusion protein of the invention may be introduced into a transgenic cell or host cell using methods known in the art, including, but not limited to electroporation, microinjection, lipid-mediated transfection (e.g., liposomal delivery systems), calcium phosphate-mediated transfection, DEAE dextran-mediated transfection, DNA transfection by biolistics, DNA transfection mediated by polybrene, and virus-mediated transduction.

Any type of cell or host cell can be used in accordance with this invention, including, but not limited to, a mammalian cell (e.g., a human, mouse, rat, monkey, or rabbit cell), a yeast cell, a bacterial cell, or an insect cell. A mammalian cell that expresses a fusion protein of the invention may include a primary cell such as a fibroblast, an epithelial cell, an endothelial cell, a smooth muscle cell, a hepatocyte, a kidney cell, and a lymphocyte. Additional examples of suitable mammalian cell lines include COS-7 monkey kidney cells, CV-1, L-cells, C127 cells, 3T3 cells, Chinese hamster ovary (CHO) cells, human embryonic kidney (HEK) cells, HeLa cells (e.g., HeLa S3 or HeLa Kyoto cells), 293 cells, 293T cells, N2A, U2OS, HUH7 and BHK cell lines. A variety of cells are commercially available for the expression of recombinant proteins, including, but not limited to, bacterial competent cells (e.g., BL21-AI™ ONE SHOT® cells, ONE SHOT®-BL21(DE3) cells, and ONE SHOT®-BL21(DE3) pLysE cells, (Invitrogen); and mammalian competent cells (e.g., MAXPAK Competent HeLa S3 cells, MAXPAK Competent CHO-K1 cells, and MAXPAK Competent HEK 293 cells (Genlantis)).

A transgenic cell that contains a nucleic acid molecule encoding the fusion protein of this invention may a stable cell line (e.g., a cell that has integrated the nucleic acid molecule encoding the fusion protein into one or more of its chromosomes). Alternatively, a transgenic cell may contain the nucleic acid molecule encoding the fusion protein in a plasmid or on an artificial chromosome, which replicates independently of the chromosomes of the cell.

A transgenic mammal may also be produced from a transgenic cell containing a nucleic acid molecule encoding the fusion protein of this invention. A transgenic animal may be a mouse, a rat, a bovine, an ovine, a caprine, a porcine, a horse, a rabbit, or a monkey. Methods for the production of a transgenic mammal from a transgenic cell are known in the art and include, without limitation, methods that require the transfer of a nucleus from a transgenic cell to an enucleated oocyte and/or the microinjection of one or more nucleic acids (e.g., a plasmid or an artificial chromosome) encoding the fusion proteins into an oocyte. Such genetically manipulated oocytes may then be transferred into a recipient female host to produce a transgenic mammal.

To facilitate the analysis of stress granule formation, this invention also provides a kit containing a nucleic acid, vector, and/or host cell encoding a fusion composed of an inducible multimerization moiety at the amino terminus, and a G3BP. The kit may further contain materials describing the kit components and instructions for using the kit components. In addition, the kit can include reagents to, e.g., insert the nucleic acid molecule into a vector (e.g., restriction enzymes or ligase), introduce the vector into a host cell (e.g., transfection reagents), and/or amplify cells (e.g., growth medium).

As is known in the art, stress granules are dense aggregates in the cytosol composed of proteins and RNAs that appear when the cell is under stress. Stress granules contain polyadenylated RNA, small ribosomal subunits, translation initiation factors (eIF3, eIF4E, eIF4G), and RNA binding proteins (RBPs) such as TIA-1, HuR, PABP, G3BP and TTP that form following eIF2α phosphorylation. Given the responsiveness of the fusion protein disclosed herein to an exogenous stimulus, this invention also provides a method for inducing stress granule formation in a cell expressing an IDM-G3BP fusion protein (e.g., a fusion protein composed of an inducible multimerization moiety at the amino terminus, and a G3BP lacking an N-terminal NTF2-like domain) in a cell and exposing the cell expressing the fusion protein to an exogenous stimulus so that stress granule formation in a cell is induced. In some embodiments, the cell is exposed to a chemical, e.g., FK506, rapamycin, iRap, AP21967, AP23102, ABA, GA₃-AM or GA₃, as the exogenous stimulus. In other embodiments, the cell is exposed to light or a magnetic field as the exogenous stimulus. In certain embodiments, the cell is exposed to light in the range of 365 to 550 nm, or more preferably in the range of 390 to 480 nm.

This invention is of particular use in the analysis of stress granules involvement in diseases such as neurodegenerative disease, cancer and infectious disease. In this respect, the protein, nucleic acids, vectors, cells and method of this find use as basic research tools as well as in screening assays for compounds that modulate stress granule formation, assembly, disassembly, or nucleation; and/or ameliorate or treat a stress granule-related disease or disorder. For example, a cell expressing a fusion protein of this invention is treated with a library of compounds, exposed to an exogenous stimulus to induce stress granule assembly and formation/localization of stress granules is measured to determine whether one or more compounds modulate the assembly or location of stress granules. Localization of the fusion protein may be measured using, e.g., an antibody that specifically binds the IDM or G3BP of the fusion protein or by fluorescence microscopy. An increase in the number of foci containing the fusion protein (e.g., intense immunostaining in distinct cellular structures) indicates an increase in the formation of stress granules. A decrease in the number of foci containing the fusion protein, likewise, indicates a decrease in the formation of stress granules. Agents that allow for the specific up-regulation of stress granule formation in cells are of use in providing increased resistance to toxic stress in a mammalian cell (e.g., for cell replacement therapies).

The following non-limiting examples are provided to further illustrate the present invention.

### Example 1: Fusion of Wild-Type G3BP1 (G3BP1_{FL}) with the Photolyase Homology Region of CRY2 (CRY_{PHR}) Leads to Stress Granule Formation

N-terminal photolyase homology region (PHR) of Arabidopsis thaliana cryptochrome 2 (CRY2) simultaneously oligomerize upon blue light stimulation (Bugaj, et al. (2013) Nature Methods 10:249; Kennedy, et al. (2000) Nature Methods 7:973-5). Expression of CRY2_{PHR}-mCherry alone in mammalian cells induces negligible visible cluster after blue light activation (Lee, et al. (2014) Nature Methods 11:633-636). Fusing Intrinsically Disordered (IDR) proteins to CRY2 causes reversible droplets in living cells upon blue light stimulation (Shin, et al. (2017) Cell 168:159-171). This system, termed OptoDroplets, creates membraneless organelles by switching on light-activated-proteins. Initially, it was determined whether OptoDroplets of FUS and TDP43 could incorporate the stress granule component G3BP1 into the droplets. This analysis indicated that G3BP1 could not be incorporated into the FUS and TDP43 Optodroplets. Moreover, OptoDroplets of FUS and TDP43 were not positive for another stress granules marker PABPC1. This indicated the OptoFUS and OptoTDP43 were not stress granules.

Accordingly, the PHR domain of CRY2 fused to mCherry (CRY2_{PHR}-mCherry) was PCR-amplified from plasmid pCRY2 PHR-mCherryN1 (Addgene) and fused to the N-terminus of full length G3BP1 (G3BP1_{FL}; ASU Biodesign) and stress granule formation by blue light induction as assessed. This analysis indicated that the CRY2_{PHR}-mCherry-G3BP1_{FL} fusion protein could form granules with blue light. Moreover, the resulting stress granules stained positive for the stress granules marker PABPC1.

### Example 2: Replacement of NTF2-Like Domain of G3BP1 (G3BP1_{D1-142}) with CRY_{PHR} Leads to Stress Granule Formation

G3BP is essential for stress granules assembly as condensate (Kedersha, et al. (2016) J. Cell Biol. 212:845). The NTF2-like domain of G3BP1 contributes to the stress granules formation by mediating oligomerization and mutual interaction with USP10 and Caprin1 (Kedersha, et al. (2016) J. Cell Biol. 212:845; Tourrière, et al. (2003) J. Cell Biol. 160:823). To reconstitute stress granules with a light inducible system, the NTF2-like domain of G3BP1 was deleted (residues 1-142; G3BP1_{D1-142}) and replaced with mCherry-tagged CRY2_{PHR}.

It has been reported that CRY2_{PHR} alone shows some nuclear bodies and little cytoplasm clustering upon blue light stimulation, while the CRY2_{PHR} E490G (CRY2_{olig}) rapidly forms light-dependent clusters (Lee, et al. (2014) Nature Methods 11:633-636; Shin, et al. (2017) Cell 168:159-171; Taslimi, et al. (2014) Nat. Commun. 5:4925). Consistent with previous reports, mCherry-tagged CRY2_{PHR} formed some nuclear clusters but limited cytoplasmic cluster, while mCherry-tagged CRY2_{olig} underwent clusters robustly upon identical activation condition in U2OS cells. Under identical blue light activation, the CRY2_{PHR}-mCherry-G3BP1_{D1-142} fusion protein could assemble into granules rapidly (in seconds). Furthermore, these granules fused to form larger granules, which disassembled in minutes after removing the stimulation. This indicates the CRY2_{PHR}-mCherry-G3BP1_{D1-142} granules were dynamic. To further elucidate the molecular dynamics of light-induced CRY2_{PHR}-mCherry-G3BP1_{D1-142} granules, fluorescence recovery was assessed after photobleaching (FRAP) experiments by photo-bleaching the mCherry signal. CRY2_{PHR}-mCherry-G3BP1_{D1-142} exhibited rapid recovery and a large mobile fraction. Taken together, these data indicate that light-dependent CRY2_{PHR}-mCherry-G3BP1_{D1-142} granules are dynamic structures.

### Example 3: CRY2_{PHR}-mCherry-G3BP1_{D1-142} Granules are Characteristic of Stress Granules

It was subsequently determined whether these CRY2_{PHR}-mCherry-G3BP1_{D1-142} granules were stress granules. First, stress granules marker GFP-TIA1 was co-expressed with the CRY2_{PHR}-mCherry-G3BP1_{D1-142} fusion protein. With blue light activation, CRY2_{PHR}-mCherry-G3BP1_{D1-142} assembled into granules and GFP-TIA1 was incorporated into these granules. As a control, it was observed that GFP-TIA1 could not be incorporated into CRY2_{olig} clusters. Another stress granules component TDP43 was also incorporated into CRY2_{PHR}-mCherry-G3BP1_{D1-142} granules. As such, the CRY2_{PHR}-mCherry-G3BP1_{D1-142} granules were positive for stress granule proteins.

Stress granules are composed of proteins and mRNA (Kedersha, et al. (2016) J. Cell Biol. 212:845; Panas, et al. (2016) J. Cell Biol. 215:313-323). To investigate whether polyadenylated mRNA were present in CRY2_{PHR}-mCherry-G3BP1_{D1-142} granules just as in canonical stress granules, FISH analysis was performed with a fluorescently conjugated oligo(dT) probe. This analysis indicated that mRNA was recruited into CRY2_{PHR}-mCherry-G3BP1_{D1-142} granules but not CRY_{FL} or CRY2_{olig} clusters after blue light stimulation. Furthermore, CRY2_{PHR}-mCherry-G3BP1_{D1-142} granules co-localized with endogenous TDP43 after photoactivation. These data indicate that photoactive CRY2_{PHR}-mCherry-G3BP1_{D1-142} granules are canonical stress granules.

### Example 4: CRY2_{PHR}-mCherry-G3BP1_{D1-142} Stress Granule Assembly is Dependent on Concentration and Blue Light Intensity

It has been reported that light-activated OptoDroplet formation shows a threshold in both concentration and light intensity (Shin, et al. (2017) Cell 168:159-171). It was contemplated that CRY2_{PHR}-mCherry-G3BP1_{D1-142} granule assembly kinetics was dependent on the local G3BP1 molecular concentration. With the CRY2 construct, the local G3BP1 molecular concentration could be controlled according to two independent methods, expression level and blue light intensity. To characterize the dynamic kinetics of CRY2_{PHR}-mCherry-G3BP1_{D1-142} stress granules, blue light intensity was continuously increased to photoactive the CRY2_{PHR}-mCherry-G3BP1_{D1-142} fusion protein beginning from weak laser power. Consistent with light-activated OptoDroplet formation, the assembly of CRY2_{PHR}-mCherry-G3BP1_{D1-142} granules was largely dependent on blue light intensity. With low blue light power, no cells could form granules. Then with double blue light power, these cells with higher expression level formed limited granules. With further increasing blue light power, more granules assembled and granules assembled in these lower expressed level cells. Furthermore, CRY2_{PHR}-mCherry-G3BP1_{D1-142} assembled quicker with higher blue light power. It was further observed that CRY2 PHR-mCherry-G3BP1_{D1-142} showed the same assembly kinetics when the blue light was saturated.

It was subsequently determined whether expression level or protein concentration contributed to assembly of CRY2_{PHR}-mCherry-G3BP1_{D1-142} granules. With fixed blue light intensity, the assembly kinetics were compared in cells with different CRY2_{PHR}-mCherry-G3BP1_{D1-142} expression levels. With the lowest expression level of CRY2 PHR-mCherry-G3BP1_{D1-142}, the cells could not form granules. The cells with higher expression levels of CRY2_{PHR}-mCherry-G3BP1_{D1-142} could form granules faster. These data indicated that the CRY2_{PHR}-mCherry-G3BP1_{D1-142} granule assembly was both concentration and blue light intensity dependent. However, it was noted that CRY2_{PHR}-mCherry-G3BP1_{D1-142} stress granule formation was independent of eIF2α phosphorylation and was in dynamic equilibrium with translating polysomes.

### Example 5: Replacement of NTF2-Like Domain of G3BP1 (G3BP1_{D1-142}) with FKBP Leads to Ligand-Inducible Stress Granule Formation

Having demonstrated that expression of CRY2-mCherry-G3BP1_{D1-142} in cells permits light-sensitive induction of stress granules in living cells, additional tools for temporal and spatial control of stress granule assembly were generated. Specifically, the human protein FKBP12 that forms dimers upon binding the small molecule ligand FK506 (Spencer, et al. (1993) Science 262:1019-1024) was used to generate the fusion construct FKBP12-mCherry-G3BP1_{D1-142} in a manner consistent with that described for CRY2_{PHR}-mCherry-G3BP1_{D1-142}. As with CRY2 and light stimulation, expression of the FKBP12-mCherry-G3BP1_{D1-142} in cells led to stress granule formation in response to ligand.

The FKBP12 protein has been re-engineered by a point mutation at amino acid residue 36 (FKBP12-F36M), which reverses ligand-dependent dimerization (Rollins, et al. (2000) Proc. Natl. Acad. Sci. USA 97:7099-7101). FKBP12-F36M forms spontaneous dimers that are disrupted by interaction with ligand. Thus, a FKBP12_{F36M}-mCherry-G3BP1_{D1-142} construct was generated and expressed in cells. Notably, it was observed that this protein forms stress granules that are disassembled by ligand, thereby providing a means of ligand-dependent disruption of stress granules.

What is claimed is:

1. A nucleic acid molecule encoding a fusion protein comprising
(a) an inducible multimerization moiety at the amino terminus of the fusion protein, and
(b) GTPase-Activating Protein SH3 Domain-Binding Protein (G3BP).

2. The nucleic acid molecule of claim 1, wherein the inducible multimerization moiety is a chemical or light inducible multimerization protein or protein domain.

3. The nucleic acid molecule of claim 2, wherein the chemical or light inducible multimerization protein or protein domain is FK506 Binding Protein (FKBP); plant cryptochrome (CRY); a light-oxygen-voltage-sensing (LOV) domain; a LOV domain-containing protein; UV-B photoreceptor; N-terminal domain of cryptochrome-interacting basic-helix-loop-helix protein (CIBN); phytochrome interacting factor (PIF); Flavin-binding, Kelch repeat, F-box 1 (FKF1); GIGANTEA, TULIPS, or Dronpa.

4. The nucleic acid of claim 3, wherein the CRY lacks a Cryptochrome C-terminal Extension (CCE) domain.

5. The nucleic acid molecule of claim 1, wherein the G3BP lacks an N-terminal Nuclear Transport Factor 2 (NTF2)-like domain.

6. The nucleic acid molecule of claim 5, wherein the G3BP comprises the amino acid sequence of SEQ ID NO:25 or SEQ ID NO:28.

7. The nucleic acid molecule of claim 1, further comprising a reporter protein.

8. A vector comprising the nucleic acid molecule of claim 1.

9. A cell harboring the vector of claim 8.

10. A method for inducing stress granule formation in a cell comprising expressing a nucleic acid molecule of claim 1 in a cell and exposing the cell to an exogenous stimulus that promotes the multimerization of the inducible multimerization domain so that stress granule formation in the cell is induced.

11. The method of claim 10, wherein the exogenous stimulus is a chemical or light.


SEQUENCE LISTING (74 sequences)

SEQ ID NO: 1 - protein, 466 aa, Homo sapiens
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 2 - protein, 482 aa, Homo sapiens
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEERQPSPEPVQENANSGYYEAHPVTNGIEEPLEESSHEPEPEPESETKTEELKPQVEEKNLEELEEKSTTPPPAEPVSLPQEPPKAFSWASVTSKNLPPSGTVSSSGIPPHVKAPVSQPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 3 - protein, 449 aa, Homo sapiens
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEERQPSPEPVQENANSGYYEAHPVTNGIEEPLEESSHEPEPEPESETKTEELKPQVEEKNLEELEEKSTTPPPAEPVSLPQEPPKPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 4 - protein, 465 aa, Rattus norvegicus
MVMEKPSPLLVGREFVRQYYTLLNQAPDMLHRFYGKNSSYAHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQSPEVVADDSGTFYDQTVSNDLEEHLEEPVVEPEPEPEPEPEPEPVSDIQEDKPEPALEEAAPEDVQKSASPAPADVAPAQEDLRTFSWASVTSKNLPPSGAVPVTGTPPHVVKVPASQPRPESKPDSQIPPQRPQRDQRAREQRINIPPQRGPRPIREAGEPGDVEPRRMVRHPDSHQLFIGNLPHEVDKSELKDFFQSYGNVVELRINSGGKLPNFGFVVFDDSEPVQKVLNNRPIMFRGAVRLNVEEKKTRAAREGDRRDNRLRGPGGPRGGPSGGMRGPPRGGMVQKPGFGVGRGITTPRQ

SEQ ID NO: 5 - protein, 465 aa, Mus musculus
MVMEKPSPLLVGREFVRQYYTLLNQAPDMLHRFYGKNSSYAHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQTVSNDLEEHLEEPVVEPEPEPEPEPEPEPVSDIQEDKPEAALEEAAPDDVQKSTSPAPADVAPAQEDLRTFSWASVTSKNLPPSGAVPVTGTPPHVVKVPASQPRPESKPDSQIPPQRPQRDQRVREQRINIPPQRGPRPIREAGEPGDVEPRRMVRHPDSHQLFIGNLPHEVDKSELKDFFQNFGNVVELRINSGGKLPNFGFVVFDDSEPVQKVLSNRPIMFRGAVRLNVEEKKTRAAREGDRRDNRLRGPGGPRGGPSGGMRGPPRGGMVQKPGFGVGRGITTPRQ

SEQ ID NO: 6 - protein, 466 aa, Macaca mulatta
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDTQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQNYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 7 - protein, 466 aa, Pan troglodytes
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDVEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 8 - protein, 465 aa, Bos taurus
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFITEPQEESEEEVEEPEERQQTPEVVPDDSGTFYDQTVSNDLEEHLEEPVAEPEPEPEPEPEQEPVSEVQEEKSEPVLEETAPEDVQKSSSPAPADIAQTVQEDLRTFSWASVTSKNLPPSGAVPVTGIPPHVVKVPASQPRPESKPESQIPLQRPQRDQRVREQRINVPPQRGPRPVREAGEQGDVEPRRIVRHPDSHQLFIGNLPHEVDKSELKDFFQNYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRSIAPRQ

SEQ ID NO: 9 - protein, 465 aa, Canis lupus
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQSVSNDLEEHLEEPVAEPEPDPEPEPEQEPVSEIQEEKSEPVLEETAPEDTQKSSSPAPTDIAQTVQEDLRTFSWASVTSKNLPPSGAVPVTGIPPHVVKVPASQPRPESKPESQIPPQRPQRDQRVREQRINIPPQRGPRPIREAGEQGDVEPRRIVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPSRGGMVQKPGFGVGRGIAPRQ

SEQ ID NO: 10 - protein, 482 aa, Mus musculus
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEDRQPSPEPVQENANSAYYDAHPVTNGIEEPLEESSHEPEPEPESETKTEELKPQVEEKHLEELEEKSATPPPAEPASLPQEPPKAFSWASVTSKNLPPSGTVSSSGIPPHVKAPVSQPRVDAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMTQKLGSGRGTGQMEGRFTGQRR

SEQ ID NO: 11 - protein, 449 aa, Mus musculus
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEDRQPSPEPVQENANSAYYDAHPVTNGIEEPLEESSHEPEPEPESETKTEELKPQVEEKHLEELEEKSATPPPAEPASLPQEPPKPRVDAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMTQKLGSGRGTGQMEGRFTGQRR

SEQ ID NO: 12 - protein, 449 aa, Rattus norvegicus
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEDRQPSPEPVQENANSAYYEAHPVTNGIEEPLEESSHEPEPEPESETKTEELKPQAEEKHLEELEEKSATPPPTEPASLPQEPPKPRVDAKPEVQSQPPRVREQRPRERPGFPPRGPRPGRGDTEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMTQKLGSGRGTGQMEGRFTGQRR

SEQ ID NO: 13 - protein, 482 aa, Rattus norvegicus
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEDRQPSPEPVQENANSAYYEAHPVTNGIEEPLEESSHEPEPEPESETKTEELKPQAEEKHLEELEEKSATPPPTEPASLPQEPPKAFSWASVTSKNLPPSGTVSSSGIPPHVKAPVSQPRVDAKPEVQSQPPRVREQRPRERPGFPPRGPRPGRGDTEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMTQKLGSGRGTGQMEGRFTGQRR

SEQ ID NO: 14 - protein, 449 aa, Bos taurus
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEERQPSPEPVQENANSGYYEAHPVTNGIEEPLEESSHEPEPEPESETKTEELKPPVEEKNLEELEEKSASPPPAEPVSLPQEPPKPRVEAKPEVQSQPPRVREQRPRERPGFPPRGPRPGRGDIEQNESDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRSDRGPGGPRGIVGGGMMRDRDGRGPPPRGGMAQKLGSGRGAGQMEGRFTGQRR

SEQ ID NO: 15 - protein, 482 aa, Bos taurus
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEERQPSPEPVQENANSGYYEAHPVTNGIEEPLEESSHEPEPEPESETKTEELKPPVEEKNLEELEEKSASPPPAEPVSLPQEPPKAFSWASVTSKNLPPSGTVSSSGIPPHVKAPVSQPRVEAKPEVQSQPPRVREQRPRERPGFPPRGPRPGRGDIEQNESDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRSDRGPGGPRGIVGGGMMRDRDGRGPPPRGGMAQKLGSGRGAGQMEGRFTGQRR

SEQ ID NO: 16 - protein, 449 aa, Canis lupus
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEERQPSPEPVQENADSGYYEAHPVANGIEEPLEESSHEPEPEPDSETKTEELKPQVEEKNLEELEEKSTSPPPAEPVSLPQEPPKPRVEAKPEVQSQPPRVREQRPRERPGFPPRGPRPGRGDLEQNESDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 17 - protein, 482 aa, Canis lupus
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEERQPSPEPVQENADSGYYEAHPVANGIEEPLEESSHEPEPEPDSETKTEELKPQVEEKNLEELEEKSTSPPPAEPVSLPQEPPKAFSWASVTSKNLPPSGTVSSSGIPPHVKAPVSQPRVEAKPEVQSQPPRVREQRPRERPGFPPRGPRPGRGDLEQNESDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 18 - protein, 449 aa, Macaca mulatta
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEERQPSPEPVQENANSGYYEAHPVTNGIEEPLEESSHEPEPETESETKTEELKPQVEEKNLEELEEKSTTPPPAEPVSLPQEPPKPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 19 - protein, 449 aa, Pan troglodytes; identical to SEQ ID NO: 3

SEQ ID NO: 20 - protein, 482 aa, Pan troglodytes; identical to SEQ ID NO: 2

SEQ ID NO: 21 - protein, 482 aa, Macaca mulatta
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEERQPSPEPVQENANSGYYEAHPVTNGIEEPLEESSHEPEPETESETKTEELKPQVEEKNLEELEEKSTTPPPAEPVSLPQEPPKAFSWASVTSKNLPPSGTVSSSGIPPHVKAPVSQPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 22 - protein, 482 aa, Artificial sequence
  Synthetic polypeptide
  MISC_FEATURE (243)..(243): X is present or absent and when present denotes Ala
  MISC_FEATURE (244)..(244): X is present or absent and when present denotes Phe
  MISC_FEATURE (245)..(245): X is present or absent and when present denotes Ser
  MISC_FEATURE (246)..(246): X is present or absent and when present denotes Trp
  MISC_FEATURE (247)..(247): X is present or absent and when present denotes Ala
  MISC_FEATURE (248)..(248): X is present or absent and when present denotes Ser
  MISC_FEATURE (249)..(249): X is present or absent and when present denotes Val
  MISC_FEATURE (250)..(250): X is present or absent and when present denotes Thr
  MISC_FEATURE (251)..(251): X is present or absent and when present denotes Ser
  MISC_FEATURE (252)..(252): X is present or absent and when present denotes Lys
  MISC_FEATURE (253)..(253): X is present or absent and when present denotes Asn
  MISC_FEATURE (254)..(254): X is present or absent and when present denotes Leu
  MISC_FEATURE (255)..(255): X is present or absent and when present denotes Pro
  MISC_FEATURE (256)..(256): X is present or absent and when present denotes Pro
  MISC_FEATURE (257)..(257): X is present or absent and when present denotes Ser
  MISC_FEATURE (258)..(258): X is present or absent and when present denotes Gly
  MISC_FEATURE (259)..(259): X is present or absent and when present denotes Thr
  MISC_FEATURE (260)..(260): X is present or absent and when present denotes Val
  MISC_FEATURE (261)..(261): X is present or absent and when present denotes Ser
  MISC_FEATURE (262)..(262): X is present or absent and when present denotes Ser
  MISC_FEATURE (263)..(263): X is present or absent and when present denotes Ser
  MISC_FEATURE (264)..(264): X is present or absent and when present denotes Gly
  MISC_FEATURE (265)..(265): X is present or absent and when present denotes Ile
  MISC_FEATURE (266)..(266): X is present or absent and when present denotes Pro
  MISC_FEATURE (267)..(267): X is present or absent and when present denotes Pro
  MISC_FEATURE (268)..(268): X is present or absent and when present denotes His
  MISC_FEATURE (269)..(269): X is present or absent and when present denotes Val
  MISC_FEATURE (270)..(270): X is present or absent and when present denotes Lys
  MISC_FEATURE (271)..(271): X is present or absent and when present denotes Ala
  MISC_FEATURE (272)..(272): X is present or absent and when present denotes Pro
  MISC_FEATURE (273)..(273): X is present or absent and when present denotes Val
  MISC_FEATURE (274)..(274): X is present or absent and when present denotes Ser
  MISC_FEATURE (275)..(275): X is present or absent and when present denotes Gln
MVMEKPSPLLVGREFVRQYYTLLNKAPEYLHRFYGRNSSYVHGGVDASGKPQEAVYGQNDIHHKVLSLNFSECHTKIRHVDAHATLSDGVVVQVMGLLSNSGQPERKFMQTFVLAPEGSVPNKFYVHNDMFRYEDEVFGDSEPELDEESEDEVEEEQEERQPSPEPVQENANSGYYEAHPVTNGIEEPLEESSHEPEPEPESETKTEELKPQVEEKNLEELEEKSTTPPPAEPVSLPQEPPKXXXXXXXXXXXXXXXXXXXXXXXXXXXXXXXXXPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 23 - protein, 6 aa, Artificial Sequence
  Synthetic peptide
LFIGNL

SEQ ID NO: 24 - protein, 8 aa, Artificial Sequence
  Synthetic peptide
PNFGFVVF

SEQ ID NO: 25 - protein, 324 aa, Homo sapiens
TEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 26 - protein, 340 aa, Homo sapiens
PELDEESEDEVEEEQEERQPSPEPVQENANSGYYEAHPVTNGIEEPLEESSHEPEPEPESETKTEELKPQVEEKNLEELEEKSTTPPPAEPVSLPQEPPKAFSWASVTSKNLPPSGTVSSSGIPPHVKAPVSQPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 27 - protein, 307 aa, Homo sapiens
PELDEESEDEVEEEQEERQPSPEPVQENANSGYYEAHPVTNGIEEPLEESSHEPEPEPESETKTEELKPQVEEKNLEELEEKSTTPPPAEPVSLPQEPPKPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 28 - protein, 340 aa, Artificial Sequence
  Synthetic polypeptide
  MISC_FEATURE (101)..(101): Xaa is present or absent and when present is Ala
  MISC_FEATURE (102)..(102): Xaa is present or absent and when present is Phe
  MISC_FEATURE (103)..(103): Xaa is present or absent and when present is Ser
  MISC_FEATURE (104)..(104): Xaa is present or absent and when present is Trp
  MISC_FEATURE (105)..(105): Xaa is present or absent and when present is Ala
  MISC_FEATURE (106)..(106): Xaa is present or absent and when present is Ser
  MISC_FEATURE (107)..(107): Xaa is present or absent and when present is Val
  MISC_FEATURE (108)..(108): Xaa is present or absent and when present is Thr
  MISC_FEATURE (109)..(109): Xaa is present or absent and when present is Ser
  MISC_FEATURE (110)..(110): Xaa is present or absent and when present is Lys
  MISC_FEATURE (111)..(111): Xaa is present or absent and when present is Asn
  MISC_FEATURE (112)..(112): Xaa is present or absent and when present is Leu
  MISC_FEATURE (113)..(113): Xaa is present or absent and when present is Pro
  MISC_FEATURE (114)..(114): Xaa is present or absent and when present is Pro
  MISC_FEATURE (115)..(115): Xaa is present or absent and when present is Ser
  MISC_FEATURE (116)..(116): Xaa is present or absent and when present is Gly
  MISC_FEATURE (117)..(117): Xaa is present or absent and when present is Thr
  MISC_FEATURE (118)..(118): Xaa is present or absent and when present is Val
  MISC_FEATURE (119)..(119): Xaa is present or absent and when present is Ser
  MISC_FEATURE (120)..(120): Xaa is present or absent and when present is Ser
  MISC_FEATURE (121)..(121): Xaa is present or absent and when present is Ser
  MISC_FEATURE (122)..(122): Xaa is present or absent and when present is Gly
  MISC_FEATURE (123)..(123): Xaa is present or absent and when present is Ile
  MISC_FEATURE (124)..(124): Xaa is present or absent and when present is Pro
  MISC_FEATURE (125)..(125): Xaa is present or absent and when present is Pro
  MISC_FEATURE (126)..(126): Xaa is present or absent and when present is His
  MISC_FEATURE (127)..(127): Xaa is present or absent and when present is Val
  MISC_FEATURE (128)..(128): Xaa is present or absent and when present is Lys
  MISC_FEATURE (129)..(129): Xaa is present or absent and when present is Ala
  MISC_FEATURE (130)..(130): Xaa is present or absent and when present is Pro
  MISC_FEATURE (131)..(131): Xaa is present or absent and when present is Val
  MISC_FEATURE (132)..(132): Xaa is present or absent and when present is Ser
  MISC_FEATURE (133)..(133): Xaa is present or absent and when present is Gln
PELDEESEDEVEEEQEERQPSPEPVQENANSGYYEAHPVTNGIEEPLEESSHEPEPEPESETKTEELKPQVEEKNLEELEEKSTTPPPAEPVSLPQEPPKXXXXXXXXXXXXXXXXXXXXXXXXXXXXXXXXXPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 29 - protein, 323 aa, Rattus norvegicus
TEPQEESEEEVEEPEERQQSPEVVADDSGTFYDQTVSNDLEEHLEEPVVEPEPEPEPEPEPEPVSDIQEDKPEPALEEAAPEDVQKSASPAPADVAPAQEDLRTFSWASVTSKNLPPSGAVPVTGTPPHVVKVPASQPRPESKPDSQIPPQRPQRDQRAREQRINIPPQRGPRPIREAGEPGDVEPRRMVRHPDSHQLFIGNLPHEVDKSELKDFFQSYGNVVELRINSGGKLPNFGFVVFDDSEPVQKVLNNRPIMFRGAVRLNVEEKKTRAAREGDRRDNRLRGPGGPRGGPSGGMRGPPRGGMVQKPGFGVGRGITTPRQ

SEQ ID NO: 30 - protein, 323 aa, Mus musculus
TEPQEESEEEVEEPEERQQTPEVVPDDSGTFYDQTVSNDLEEHLEEPVVEPEPEPEPEPEPEPVSDIQEDKPEAALEEAAPDDVQKSTSPAPADVAPAQEDLRTFSWASVTSKNLPPSGAVPVTGTPPHVVKVPASQPRPESKPDSQIPPQRPQRDQRVREQRINIPPQRGPRPIREAGEPGDVEPRRMVRHPDSHQLFIGNLPHEVDKSELKDFFQNFGNVVELRINSGGKLPNFGFVVFDDSEPVQKVLSNRPIMFRGAVRLNVEEKKTRAAREGDRRDNRLRGPGGPRGGPSGGMRGPPRGGMVQKPGFGVGRGITTPRQ

SEQ ID NO: 31 - protein, 323 aa, Bos taurus
TEPQEESEEEVEEPEERQQTPEVVPDDSGTFYDQTVSNDLEEHLEEPVAEPEPEPEPEPEQEPVSEVQEEKSEPVLEETAPEDVQKSSSPAPADIAQTVQEDLRTFSWASVTSKNLPPSGAVPVTGIPPHVVKVPASQPRPESKPESQIPLQRPQRDQRVREQRINVPPQRGPRPVREAGEQGDVEPRRIVRHPDSHQLFIGNLPHEVDKSELKDFFQNYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRSIAPRQ

SEQ ID NO: 32 - protein, 324 aa, Macaca mulatta
TEPQEESEEEVEEPEERQQTPEVVPDDSGTFYDQAVVSNDMEEHLEEPVAEPEPDPEPEPEQEPVSEIQEEKPEPVLEETAPEDTQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQNYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 33 - protein, 324 aa, Pan troglodytes
TEPQEESEEEVEEPEERQQTPEVVPDDSGTFYDQAVVSNDVEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 34 - protein, 323 aa, Canis lupus
TEPQEESEEEVEEPEERQQTPEVVPDDSGTFYDQSVSNDLEEHLEEPVAEPEPDPEPEPEQEPVSEIQEEKSEPVLEETAPEDTQKSSSPAPTDIAQTVQEDLRTFSWASVTSKNLPPSGAVPVTGIPPHVVKVPASQPRPESKPESQIPPQRPQRDQRVREQRINIPPQRGPRPIREAGEQGDVEPRRIVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPSRGGMVQKPGFGVGRGIAPRQ

SEQ ID NO: 35 - protein, 340 aa, Mus musculus
PELDEESEDEVEEEQEDRQPSPEPVQENANSAYYDAHPVTNGIEEPLEESSHEPEPEPESETKTEELKPQVEEKHLEELEEKSATPPPAEPASLPQEPPKAFSWASVTSKNLPPSGTVSSSGIPPHVKAPVSQPRVDAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMTQKLGSGRGTGQMEGRFTGQRR

SEQ ID NO: 36 - protein, 307 aa, Mus musculus
PELDEESEDEVEEEQEDRQPSPEPVQENANSAYYDAHPVTNGIEEPLEESSHEPEPEPESETKTEELKPQVEEKHLEELEEKSATPPPAEPASLPQEPPKPRVDAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMTQKLGSGRGTGQMEGRFTGQRR

SEQ ID NO: 37 - protein, 307 aa, Rattus norvegicus
PELDEESEDEVEEEQEDRQPSPEPVQENANSAYYEAHPVTNGIEEPLEESSHEPEPEPESETKTEELKPQAEEKHLEELEEKSATPPPTEPASLPQEPPKPRVDAKPEVQSQPPRVREQRPRERPGFPPRGPRPGRGDTEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMTQKLGSGRGTGQMEGRFTGQRR

SEQ ID NO: 38 - protein, 340 aa, Rattus norvegicus
PELDEESEDEVEEEQEDRQPSPEPVQENANSAYYEAHPVTNGIEEPLEESSHEPEPEPESETKTEELKPQAEEKHLEELEEKSATPPPTEPASLPQEPPKAFSWASVTSKNLPPSGTVSSSGIPPHVKAPVSQPRVDAKPEVQSQPPRVREQRPRERPGFPPRGPRPGRGDTEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMTQKLGSGRGTGQMEGRFTGQRR

SEQ ID NO: 39 - protein, 307 aa, Canis lupus
PELDEESEDEVEEEQEERQPSPEPVQENADSGYYEAHPVANGIEEPLEESSHEPEPEPDSETKTEELKPQVEEKNLEELEEKSTSPPPAEPVSLPQEPPKPRVEAKPEVQSQPPRVREQRPRERPGFPPRGPRPGRGDLEQNESDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 40 - protein, 340 aa, Canis lupus
PELDEESEDEVEEEQEERQPSPEPVQENADSGYYEAHPVANGIEEPLEESSHEPEPEPDSETKTEELKPQVEEKNLEELEEKSTSPPPAEPVSLPQEPPKAFSWASVTSKNLPPSGTVSSSGIPPHVKAPVSQPRVEAKPEVQSQPPRVREQRPRERPGFPPRGPRPGRGDLEQNESDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 41 - protein, 307 aa, Bos taurus
PELDEESEDEVEEEQEERQPSPEPVQENANSGYYEAHPVTNGIEEPLEESSHEPEPEPESETKTEELKPPVEEKNLEELEEKSASPPPAEPVSLPQEPPKPRVEAKPEVQSQPPRVREQRPRERPGFPPRGPRPGRGDIEQNESDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRSDRGPGGPRGIVGGGMMRDRDGRGPPPRGGMAQKLGSGRGAGQMEGRFTGQRR

SEQ ID NO: 42 - protein, 340 aa, Bos taurus
PELDEESEDEVEEEQEERQPSPEPVQENANSGYYEAHPVTNGIEEPLEESSHEPEPEPESETKTEELKPPVEEKNLEELEEKSASPPPAEPVSLPQEPPKAFSWASVTSKNLPPSGTVSSSGIPPHVKAPVSQPRVEAKPEVQSQPPRVREQRPRERPGFPPRGPRPGRGDIEQNESDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRSDRGPGGPRGIVGGGMMRDRDGRGPPPRGGMAQKLGSGRGAGQMEGRFTGQRR

SEQ ID NO: 43 - protein, 340 aa, Pan troglodytes; identical to SEQ ID NO: 26

SEQ ID NO: 44 - protein, 340 aa, Macaca mulatta
PELDEESEDEVEEEQEERQPSPEPVQENANSGYYEAHPVTNGIEEPLEESSHEPEPETESETKTEELKPQVEEKNLEELEEKSTTPPPAEPVSLPQEPPKAFSWASVTSKNLPPSGTVSSSGIPPHVKAPVSQPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 45 - protein, 307 aa, Pan troglodytes; identical to SEQ ID NO: 27

SEQ ID NO: 46 - protein, 307 aa, Macaca mulatta
PELDEESEDEVEEEQEERQPSPEPVQENANSGYYEAHPVTNGIEEPLEESSHEPEPETESETKTEELKPQVEEKNLEELEEKSTTPPPAEPVSLPQEPPKPRVEAKPEVQSQPPRVREQRPRERPGFPPRGPRPGRGDMEQNDSDNRRIIRYPDSHQLFVGNLPHDIDENELKEFFMSFGNVVELRINTKGVGGKLPNFGFVVFDDSEPVQRILIAKPIMFRGEVRLNVEEKKTRAARERETRGGGDDRRDIRRNDRGPGGPRGIVGGGMMRDRDGRGPPPRGGMAQKLGSGRGTGQMEGRFTGQRR

SEQ ID NO: 47 - protein, 718 aa, Oryza sativa
MSASPSSMSGAGAGEAGVRTVVWFRRDLRVEDNPALAAAARAAGEVVPVYVWAPEEDGPYYPGRVSRWWLSQSLKHLDASLRRLGASRLVTRRSADAVVALIELVRSIGATHLFFNHLYDPLSLVRDHRVKALLTAEGIAVQSFNADLLYEPWEVVDDDGCPFTMFAPFWDRCLCMPDPAAPLLPPKRIAPGELPARRCPSDELVFEDESERGSNALLARAWSPGWQNADKALAAFLNGPLMDYSVNRKKADSASTSLLSPYLHFGELSVRKVFHQVRMKQLMWSNEGNHAGDESCVLFLRSIGLREYSRYLTFNHPCSLEKPLLAHLRFFPWVVDEVYFKVWRQGRTGYPLVDAGMRELWATGWLHDRIRVVVSSFFVKVLQLPWRWGMKYFWDTLLDADLESDALGWQYISGSLPDGRELDRIDNPQLEGYKFDPHGEYVRRWLPELARLPTEWIHHPWDAPESVLQAAGIELGSNYPLPIVELDAAKTRLQDALSEMWELEAASRAAMENGMEEGLGDSSDVPPIAFPPELQMEVDRAPAQPTVHGPTTAGRRREDQMVPSMTSSLVRAETELSADFDNSMDSRPEVPSQVLFQPRMEREETVDGGGGGGMVGRSNGGGHQGQHQQQQHNFQTTIHRARGVAPSTSEASSNWTGREGGVVPVWSPPAASGPSDHYAADEADITSRSYLDRHPQSHTLMNWSQLSQSLTTGWEVEN

SEQ ID NO: 48 - protein, 690 aa, Sorghum bicolor
MSASSSSLCGGDPAMRSVVWFRRDLRVEDNPALAAAARAGGEVVPAYVWSPEEEGPYYPGRVSRWWISQSLKRLDASLRRLGAGKLVTRRSADAVVALLQLVRDTGATHVYFNHLYDPISLVRDHRLKEMLAAEGIVVQSFNADLLYEPWEVVDDEGQPFTMFTAFWNRCLSMQYDPPAPLLPPKKINSGDLSMCPSEDLIFEDDSERGSNALLARAWTPGWQNADKALTAFLNGPLADYSVNRKKADSASTSLLSPHLHFGELSVRKVFHLVRMKQLVWSNEGNHAAEESCTLFLRSIGLREYSRYLSFNHPSSHERPLLAHLRFFPWVVNESYFKIWRQGRTGYPLVDAGMRELWATGWLHDRIRVVVSSFFVKVLQLPWRWGMKYFWDTLLDADLESDALGWQYITGSLPDSRELDRIDNPQFEGYKFDPHGEYVRRWIPELVRLPTEWIHHPWDAPVSVLQAAGIELGSNYPLPIVELDAAKARLQEALSEMWQLEAASRATMNNGMEEGLGDSSEVPFPEELQMEVDRATANVVMTVRRREDQMVPTMTSSLNRAETEVSADLGNSEDTRAQVPFHAHFHPRVEREDMIQNTEGPALRINGTHQHNIFQQPQNHRREALAPSVSEASSSWTGREGAVVPVWSPPAASGHSETFAADEADVSSRSYLDRHPRSYRLMNWSQLSQSL

SEQ ID NO: 49 - protein, 681 aa, Arabidopsis thaliana
MSGSVSGCGSGGCSIVWFRRDLRVEDNPALAAAVRAGPVIALFVWAPEEEGHYHPGRVSRWWLKNSLAQLDSSLRSLGTCLITKRSTDSVASLLDVVKSTGASQIFFNHLYDPLSLVRDHRAKDVLTAQGIAVRSFNADLLYEPWEVTDELGRPFSMFAAFWERCLSMPYDPESPLLPPKKIISGDVSKCVADPLVFEDDSEKGSNALLARAWSPGWSNGDKALTTFINGPLLEYSKNRRKADSATTSFLSPHLHFGEVSVRKVFHLVRIKQVAWANEGNEAGEESVNLFLKSIGLREYSRYISFNHPYSHERPLLGHLKFFPWAVDENYFKAWRQGRTGYPLVDAGMRELWATGWLHDRIRVVVSSFFVKVLQLPWRWGMKYFWDTLLDADLESDALGWQYITGTLPDSREFDRIDNPQFEGYKFDPNGEYVRRWLPELSRLPTDWIHHPWNAPESVLQAAGIELGSNYPLPIVGLDEAKARLHEALSQMWQLEAASRAAIENGSEEGLGDSAEVEEAPIEFPRDITMEETEPTRLNPNRRYEDQMVPSITSSLIRPEEDEESSLNLRNSVGDSRAEVPRNMVNTNQAQQRRAEPASNQVTAMIPEFNIRIVAESTEDSTAESSSSGRRERSGGIVPEWSPGYSEQFPSEENGIGGGSTTSSYLQNHHEILNWRRLSQTG

SEQ ID NO: 50 - protein, 679 aa, Lycopersicon esculentum
MSGGGCSIVWFRRDLRVEDNPALAAGVRAGAVIAVFIYAPEEEGHYYPGRVSRWWLKQSLAHLDSSLKSLGTSLITKRSTDSISSLLEVVKSTGATQLFFNHLYDPISLVRDHRTKEILTAQGISVRSFNADLLYEPWEVNDDEGRPFTTFSAFWEKCLSMPYDPEAPLLPPKRIISGDASRCPSDNLVFEDESEKGSNALLARAWSPGWSNADKALTTFVNGPLLEYSQNRRKADSATTSFLSPHLHFGEVSVRKVFHFVRIKQVLWANEGNKAGEESVNLFLKSIGLREYSRYMSFNHPYSHERPLLGHLRYFPWVVDEGYFKAWRQGRTGYPLVDAGMRELWATGWLHDRIRVVVSSFFVKVLQLPWRWGMKYFWDTLLDADLESDALGWQYISGTLPDGRELDRIDNPQFVGYKCDPHGEYVRRWLPELARLPTEWIHHPWNAPESVLEAAGIELGSNYPLPIVEIDSAKVRLEQALSQMWQNDAAARAAIENGMEEGHGDSADSPIAFPQAMHMEMDHEPVRNNPVIVTVRRYEDQMVPSMTSSLFRAEDEENSVDIRNSVVESRAEVPTDINVAEVHRRDTRDQAVMQTARTNATPHFNFAVGRRNSEDSTAESSSSTRERDGGVVPTWSPSSSNYSDQYVGDDNGIGTSSSYLQRHPQSHQLMNWQRLSQTG

SEQ ID NO: 51 - protein, 681 aa, Glycine max
MSGGGCSIVWFRRDLRVEDNPALAAGVRAGAVISVFIWAPEEEGQYYPGRVSRWWLKQSLAHLDSSLRNLGSPLITKRSTNSISSLLEVVKSTGATQLFFNHLYDPLSLVRDHRAKEVLTAQGITVRSFNSDLLYEPWDVNDAHGQPFTTFSAFWERCLSMPYDPQAPLLPPKRIIPGDVPRCPSDTLVFEDELEKASNALLARAWSPGWSNADKALTAFVNGALIEYSKNRRKADSATTSFLSPHLHFGEVSVKKVFHLVRIKQVFWANEGNKAGEESVNLFLKSIGLREYSRYISFNHPYSHERPLLAHLKFFPWVVNEGYFKAWRQGRTGYPLVDAGMRELWATGWLHDRIRVVVSSFFVKVLQLPWRWGMKYFWDTLLDADLESDALGWQYISGTLPDGRELDRIDNPQFEGYKCDPNGEYVRRWLPELARLPTEWIHHPWNAPESVLQAAGIELGSNYPLPIVGIDAAEVRLQEALIQMWQQEAASRAAMENGTEEGLGDSAESAPIAFPQDIQMEERPEPVRNNLPHGTRRYQDQMVPSITSSHVRVEEEETSSDLRNSAADSRAEVPINVTTQQIARETVNQGVLLNANRNTRVQNNATTWLRNAAEDSTAESSSSTRRERDGGVVPVWSPPASNFSEQFVDDENGIGAGSSYLQRQHPQSHQLMNWTRLPQTG

SEQ ID NO: 52 - protein, 727 aa, Physcomitrella patens
MAACTIVWFRRDLRLEDNPALIAAARAGTVVPVFVWSPAEDGQFHPGRVSRWWLKQSLTHLELSLKKLGSPLILRKSPDTLSVLLEIAEATGATQVFYNHLYDPVSLVRDHRVKQGLSQRGIVVHTFNGDLLYEPWEVYDEEGQAFTVYEAFWKKCMSMPFEPEAPLLPPRRLTGPIGKIVGCNAEELGLEDEFEKSSNALLARAWCPGWGFANKSLDSFLRSPLIDYARDRQKADGASGTPTSLLSPHLHFGELSVRKIFHEVRKRQITWAREGNAGGEASVNMFLRALGFREYSRYLSFHFPFTHERSLLANLKSFPWRADEGYFKAWRQGRTGYPLVDAGMRELWATGWAHNRIRVVVASFSVKFLQLPWRWGMKYFWDVLLDADLECDVLGWQYISGSLPDGHELDRIENPEVEGYRFDPDGDYVRRWIPELARLPNEWVHHPWDAPPSALRAAGVELGTNYPRPIVEIGAARERLQASLAEMWERDAAMKAALANGLEEGLGETVEVAGTGGPEHERMDVPRVMVHMQRDADMSCNSSRRDQLVPEIVPNQFHIRAHESIMNRSAAMVEDGEEAGRAAVPMVFASVRRGMGGNYGGHHVEGNGGEVAQASAPIQWPTVTAVDYELDSTAESASVTGRGGSEGGTVPVWSQSVSARTPIQVREGLVPEVRRGPGLSRRQLQASVQRVNLEGMTSNKQAEEEDFYVPKLVKWTQPRKRRVKQDG

SEQ ID NO: 53 - protein, 654 aa, Sorghum bicolor
MAGSGKTVVWFRRDLRIHDNPALAAAAKEGSVLPLFIWCPADYEQYYPGRCSRWWLKQSLAHLGKSLELLGCPLVLIRAEDSTLATLLECVHCISATRVVYNRLYDPISLVLDDKIKNELSAHGISVQSFNGDLLYEPWDVYDENGQAFTSFNKYWEKCMNVPIEISQYLAPTRLVAAPGLANVRCCSIDDLGLESSKDVESSNALLSRAWSPGWRNAENMLEEFLSCGLLEYSKHGMKVGGTTTSLLSPYLHFGELSVRKVYQLVTMHHVKWQNEGKSEAEESVRLFLRSIGFREYSRYLCFNFPFTHERSFLGNLKHYPWLLDEDRFKSWRQGMTGYPLVDAGMRELWATGWTHNRIRVIVSSFAVKCLQIPWIWGMKYFWDVLLDADLESDILGWQYISGSLPDGHELSRLDNPEVQGQKYDPDGEYVRTWIPELARMPTEWIHCPWSAPNSILQVAGVELGFNYPKPIVELHMARECLDDAISTMWQLDTAAKLAALDGEVVDDNLNNIRSFDIPKVVLKKKLSPSTSSMNKRVLSTNGKNEKSQPTEVKAPYKQIIRDDMINASNMDDTGSTANLQVTRKRSRSDSAFNVPSSSSSLVMESRIHDNDSCSVRYSGYLQQTADRDDTDKVEDNDSEDSGTSISRPSKRPA

SEQ ID NO: 54 - protein, 651 aa, Oryza sativa
MAGSERTVVWFRRDLRIDDNPALASAARDGAVLPVFIWCPADEGQFYPGRCSRWWLKQSLPHLSQSLESLGCPLVLIRAESTLEALLRCIDSVGATRLVYNHLYDPVSLVRDDKIKKELSALGISIQSFNGDLLYEPWEIYDDSGLAFTTFNMYWEKCMELPIDASPSLAPWKLVPVPGLESVRSCSVDDLGLESSKDEESSNALLMRAWSPGWRNAEKMLEEFVSHGLLEYSKHGMKVEGATTSLLSPYLHFGEVSVRKVYQLVRMQQIKWENEGTSEAEESIHFFMRSIGLREYSRYLCFNFPFTHEKSLLGNLKHYPWKVDEERFKSWRQGMTGYPLVDAGMRELWATGWTHNRIRVIISSFAVKFLLIPWTWGMKYFWDVLLDADLESDILGWQYISGSLPDGHELSRLDNPEVQGQKYDPDGVYVRTWIPELARMPTEWIHHPWDAPSCILEVAGVELGFNYPKPIVDLHIARECLDDSISTMWQLDTAEKLAELDGEVVEDNLSNIKTFDIPKVVLRETSPCALPIDQRVPHASSKDHNLKSKVLKASNRSSICVDMIRSSKMEATSSVANSPVSRKRSFCETAFHVPSYSSSAEVHSHIQDHGGSLVGPSRYLLQEAGRNYVDEVEDSSTADSGSSISRQRKAA

SEQ ID NO: 55 - protein, 612 aa, Arabidopsis thaliana
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAPDEIVADSFEALGANTIKEPGLCPSVSSNDQQVPSAVRYNGSKRVKPEEEEERDMKKSRGFDERELFSTAESSSSSSVFFVSQSCSLASEGKNLEGIQDSSDQITTSLGKNGCK

SEQ ID NO: 56 - protein, 634 aa, Glycine max
MGSNRTIVWFRRDLRIEDNPALTAAAKEGSVLPVYVWCPKEEGQFYPGRVSRWWLKQSLAHLDQSLKSLGSRLVLIKTHSTAVALVECVKAIQATKVVFNHLYDPVSLVRDHNIKEKLVEQGISVQSYNGDLLYEPWEVNSESGRAFTTFNAFWKKCLHMQMDIVSVVPPWQLIPAEGKIEECSLEELGLENESEKPSNALLGRAWSPGWRNADKALREFVELHLLHYSKKRLKVGGESTSLLSPYLHFGELSARKVFQVTCMKQILWTNEGNSAGEESANLFLRAIGLREYSRYLCFNFPFTHERALLGHLKFFPWNPDPDIFKTWRQGRTGFPLVDAGMRELWATGWIHNRIRVIVSSFAVKMLLLPWKWGMKYFWDTLLDADLESDILGWQYISGGLPDGHELERLDNPEIQGAKFDPEGEYVRQWLPELARMPTEWIHHPWDAPLTVLRAAGVELGQNYPKPIIDIDLARERLTEAIFKMWESEAAAKAAGSEPRDEVVVDNSHTVENLDTQKVVVLGKAPCATISANDQKVPALQDSKNEPPTRKRPKHMIEEGQNQDHSQNHNKDTGLSSIDQDICSTADSSSCKKQCASTSSYSFSVPQQCSSSSNLKWPWQEKIDMEQSSSKDGAM

SEQ ID NO: 57 - protein, 525 aa, Oryza sativa
MSASPSSMSGAGAGEAGVRTVVWFRRDLRVEDNPALAAAARAAGEVVPVYVWAPEEDGPYYPGRVSRWWLSQSLKHLDASLRRLGASRLVTRRSADAVVALIELVRSIGATHLFFNHLYDPLSLVRDHRVKALLTAEGIAVQSFNADLLYEPWEVVDDDGCPFTMFAPFWDRCLCMPDPAAPLLPPKRIAPGELPARRCPSDELVFEDESERGSNALLARAWSPGWQNADKALAAFLNGPLMDYSVNRKKADSASTSLLSPYLHFGELSVRKVFHQVRMKQLMWSNEGNHAGDESCVLFLRSIGLREYSRYLTFNHPCSLEKPLLAHLRFFPWVVDEVYFKVWRQGRTGYPLVDAGMRELWATGWLHDRIRVVVSSFFVKVLQLPWRWGMKYFWDTLLDADLESDALGWQYISGSLPDGRELDRIDNPQLEGYKFDPHGEYVRRWLPELARLPTEWIHHPWDAPESVLQAAGIELGSNYPLPIVELDAAKTRLQDALSEMWELEAASRAAMENGMEEGLGDSSDV

SEQ ID NO: 58 - protein, 521 aa, Sorghum bicolor
MSASSSSLCGGDPAMRSVVWFRRDLRVEDNPALAAAARAGGEVVPAYVWSPEEEGPYYPGRVSRWWISQSLKRLDASLRRLGAGKLVTRRSADAVVALLQLVRDTGATHVYFNHLYDPISLVRDHRLKEMLAAEGIVVQSFNADLLYEPWEVVDDEGQPFTMFTAFWNRCLSMQYDPPAPLLPPKKINSGDLSMCPSEDLIFEDDSERGSNALLARAWTPGWQNADKALTAFLNGPLADYSVNRKKADSASTSLLSPHLHFGELSVRKVFHLVRMKQLVWSNEGNHAAEESCTLFLRSIGLREYSRYLSFNHPSSHERPLLAHLRFFPWVVNESYFKIWRQGRTGYPLVDAGMRELWATGWLHDRIRVVVSSFFVKVLQLPWRWGMKYFWDTLLDADLESDALGWQYITGSLPDSRELDRIDNPQFEGYKFDPHGEYVRRWIPELVRLPTEWIHHPWDAPVSVLQAAGIELGSNYPLPIVELDAAKARLQEALSEMWQLEAASRATMNNGMEEGLGDSSEV

SEQ ID NO: 59 - protein, 499 aa, Arabidopsis thaliana
MSGSVSGCGSGGCSIVWFRRDLRVEDNPALAAAVRAGPVIALFVWAPEEEGHYHPGRVSRWWLKNSLAQLDSSLRSLGTCLITKRSTDSVASLLDVVKSTGASQIFFNHLYDPLSLVRDHRAKDVLTAQGIAVRSFNADLLYEPWEVTDELGRPFSMFAAFWERCLSMPYDPESPLLPPKKIISGDVSKCVADPLVFEDDSEKGSNALLARAWSPGWSNGDKALTTFINGPLLEYSKNRRKADSATTSFLSPHLHFGEVSVRKVFHLVRIKQVAWANEGNEAGEESVNLFLKSIGLREYSRYISFNHPYSHERPLLGHLKFFPWAVDENYFKAWRQGRTGYPLVDAGMRELWATGWLHDRIRVVVSSFFVKVLQLPWRWGMKYFWDTLLDADLESDALGWQYITGTLPDSREFDRIDNPQFEGYKFDPNGEYVRRWLPELSRLPTDWIHHPWNAPESVLQAAGIELGSNYPLPIVGLDEAKARLHEALSQMWQLEAASR

SEQ ID NO: 60 - protein, 509 aa, Lycopersicon esculentum
MSGGGCSIVWFRRDLRVEDNPALAAGVRAGAVIAVFIYAPEEEGHYYPGRVSRWWLKQSLAHLDSSLKSLGTSLITKRSTDSISSLLEVVKSTGATQLFFNHLYDPISLVRDHRTKEILTAQGISVRSFNADLLYEPWEVNDDEGRPFTTFSAFWEKCLSMPYDPEAPLLPPKRIISGDASRCPSDNLVFEDESEKGSNALLARAWSPGWSNADKALTTFVNGPLLEYSQNRRKADSATTSFLSPHLHFGEVSVRKVFHFVRIKQVLWANEGNKAGEESVNLFLKSIGLREYSRYMSFNHPYSHERPLLGHLRYFPWVVDEGYFKAWRQGRTGYPLVDAGMRELWATGWLHDRIRVVVSSFFVKVLQLPWRWGMKYFWDTLLDADLESDALGWQYISGTLPDGRELDRIDNPQFVGYKCDPHGEYVRRWLPELARLPTEWIHHPWNAPESVLEAAGIELGSNYPLPIVEIDSAKVRLEQALSQMWQNDAAARAAIENGMEEGHGDSADS

SEQ ID NO: 61 - protein, 510 aa, Glycine max
MSGGGCSIVWFRRDLRVEDNPALAAGVRAGAVISVFIWAPEEEGQYYPGRVSRWWLKQSLAHLDSSLRNLGSPLITKRSTNSISSLLEVVKSTGATQLFFNHLYDPLSLVRDHRAKEVLTAQGITVRSFNSDLLYEPWDVNDAHGQPFTTFSAFWERCLSMPYDPQAPLLPPKRIIPGDVPRCPSDTLVFEDELEKASNALLARAWSPGWSNADKALTAFVNGALIEYSKNRRKADSATTSFLSPHLHFGEVSVKKVFHLVRIKQVFWANEGNKAGEESVNLFLKSIGLREYSRYISFNHPYSHERPLLAHLKFFPWVVNEGYFKAWRQGRTGYPLVDAGMRELWATGWLHDRIRVVVSSFFVKVLQLPWRWGMKYFWDTLLDADLESDALGWQYISGTLPDGRELDRIDNPQFEGYKCDPNGEYVRRWLPELARLPTEWIHHPWNAPESVLQAAGIELGSNYPLPIVGIDAAEVRLQEALIQMWQQEAASRAAMENGTEEGLGDSAESA

SEQ ID NO: 62 - protein, 522 aa, Physcomitrella patens
MAACTIVWFRRDLRLEDNPALIAAARAGTVVPVFVWSPAEDGQFHPGRVSRWWLKQSLTHLELSLKKLGSPLILRKSPDTLSVLLEIAEATGATQVFYNHLYDPVSLVRDHRVKQGLSQRGIVVHTFNGDLLYEPWEVYDEEGQAFTVYEAFWKKCMSMPFEPEAPLLPPRRLTGPIGKIVGCNAEELGLEDEFEKSSNALLARAWCPGWGFANKSLDSFLRSPLIDYARDRQKADGASGTPTSLLSPHLHFGELSVRKIFHEVRKRQITWAREGNAGGEASVNMFLRALGFREYSRYLSFHFPFTHERSLLANLKSFPWRADEGYFKAWRQGRTGYPLVDAGMRELWATGWAHNRIRVVVASFSVKFLQLPWRWGMKYFWDVLLDADLECDVLGWQYISGSLPDGHELDRIENPEVEGYRFDPDGDYVRRWIPELARLPNEWVHHPWDAPPSALRAAGVELGTNYPRPIVEIGAARERLQASLAEMWERDAAMKAALANGLEEGLGETVEVAGTGGPEHER

SEQ ID NO: 63 - protein, 491 aa, Sorghum bicolor
MAGSGKTVVWFRRDLRIHDNPALAAAAKEGSVLPLFIWCPADYEQYYPGRCSRWWLKQSLAHLGKSLELLGCPLVLIRAEDSTLATLLECVHCISATRVVYNRLYDPISLVLDDKIKNELSAHGISVQSFNGDLLYEPWDVYDENGQAFTSFNKYWEKCMNVPIEISQYLAPTRLVAAPGLANVRCCSIDDLGLESSKDVESSNALLSRAWSPGWRNAENMLEEFLSCGLLEYSKHGMKVGGTTTSLLSPYLHFGELSVRKVYQLVTMHHVKWQNEGKSEAEESVRLFLRSIGFREYSRYLCFNFPFTHERSFLGNLKHYPWLLDEDRFKSWRQGMTGYPLVDAGMRELWATGWTHNRIRVIVSSFAVKCLQIPWIWGMKYFWDVLLDADLESDILGWQYISGSLPDGHELSRLDNPEVQGQKYDPDGEYVRTWIPELARMPTEWIHCPWSAPNSILQVAGVELGFNYPKPIVELHMARECLDDAISTMWQ

SEQ ID NO: 64 - protein, 482 aa, Oryza sativa
MAGSERTVVWFRRDLRIDDNPALASAARDGAVLPVFIWCPADEGQFYPGRCSRWWLKQSLPHLSQSLESLGCPLVLIRAESTLEALLRCIDSVGATRLVYNHLYDPVSLVRDDKIKKELSALGISIQSFNGDLLYEPWEIYDDSGLAFTTFNMYWEKCMELPIDASPSLAPWKLVPVPGLESVRSCSVDDLGLESSKDEESSNALLMRAWSPGWRNAEKMLEEFVSHGLLEYSKHGMKVEGATTSLLSPYLHFGEVSVRKVYQLVRMQQIKWENEGTSEAEESIHFFMRSIGLREYSRYLCFNFPFTHEKSLLGNLKHYPWKVDEERFKSWRQGMTGYPLVDAGMRELWATGWTHNRIRVIISSFAVKFLLIPWTWGMKYFWDVLLDADLESDILGWQYISGSLPDGHELSRLDNPEVQGQKYDPDGVYVRTWIPELARMPTEWIHHPWDAPSCILEVAGVELGFNYPKPIVDLHIARECLD

SEQ ID NO: 65 - protein, 498 aa, Arabidopsis thaliana
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAA

SEQ ID NO: 66 - protein, 481 aa, Glycine max
MGSNRTIVWFRRDLRIEDNPALTAAAKEGSVLPVYVWCPKEEGQFYPGRVSRWWLKQSLAHLDQSLKSLGSRLVLIKTHSTAVALVECVKAIQATKVVFNHLYDPVSLVRDHNIKEKLVEQGISVQSYNGDLLYEPWEVNSESGRAFTTFNAFWKKCLHMQMDIVSVVPPWQLIPAEGKIEECSLEELGLENESEKPSNALLGRAWSPGWRNADKALREFVELHLLHYSKKRLKVGGESTSLLSPYLHFGELSARKVFQVTCMKQILWTNEGNSAGEESANLFLRAIGLREYSRYLCFNFPFTHERALLGHLKFFPWNPDPDIFKTWRQGRTGFPLVDAGMRELWATGWIHNRIRVIVSSFAVKMLLLPWKWGMKYFWDTLLDADLESDILGWQYISGGLPDGHELERLDNPEIQGAKFDPEGEYVRQWLPELARMPTEWIHHPWDAPLTVLRAAGVELGQNYPKPIIDIDLARERLTEAI

SEQ ID NO: 67 - protein, 822 aa, Artificial Sequence
  Synthetic polypeptide
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAATEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 68 - protein, 1208 aa, Artificial Sequence
  Synthetic polypeptide
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAARDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 69 - DNA, 3630 bp, Artificial Sequence
  Synthetic polynucleotide
atgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagctttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcagcccgggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagatggtgatggagaagcctagtcccctgctggtcgggcgggaatttgtgagacagtattacacactgctgaaccaggccccagacatgctgcatagattttatggaaagaactcttcttatgtccatgggggattggattcaaatggaaagccagcagatgcagtctacggacagaaagaaatccacaggaaagtgatgtcacaaaacttcaccaactgccacaccaagattcgccatgttgatgctcatgccacgctaaatgatggtgtggtagtccaggtgatggggcttctctctaacaacaaccaggctttgaggagattcatgcaaacgtttgtccttgctcctgaggggtctgttgcaaataaattctatgttcacaatgatatcttcagataccaagatgaggtctttggtgggtttgtcactgagcctcaggaggagtctgaagaagaagtagaggaacctgaagaaagacagcaaacacctgaggtggtacctgatgattctggaactttctatgatcaggcagttgtcagtaatgacatggaagaacatttagaggagcctgttgctgaaccagagcctgatcctgaaccagaaccagaacaagaacctgtatctgaaatccaagaggaaaagcctgagccagtattagaagaaactgcccctgaggatgctcagaagagttcttctccagcacctgcagacatagctcagacagtacaggaagacttgaggacattttcttgggcatctgtgaccagtaagaatcttccacccagtggagctgttccagttactgggataccacctcatgttgttaaagtaccagcttcacagccccgtccagagtctaagcctgaatctcagattccaccacaaagacctcagcgggatcaaagagtgcgagaacaacgaataaatattcctccccaaaggggacccagaccaatccgtgaggctggtgagcaaggtgacattgaaccccgaagaatggtgagacaccctgacagtcaccaactcttcattggcaacctgcctcatgaagtggacaaatcagagcttaaagatttctttcaaagttatggaaacgtggtggagttgcgcattaacagtggtgggaaattacccaattttggttttgttgtgtttgatgattctgagcctgttcagaaagtccttagcaacaggcccatcatgttcagaggtgaggtccgtctgaatgtcgaagagaagaagactcgagctgccagggaaggcgaccgacgagataatcgccttcggggacctggaggccctcgaggtgggctgggtggtggaatgagaggccctccccgtggaggcatggtgcagaaaccaggatttggagtgggaagggggcttgcgccacggcagtgataa

SEQ ID NO: 70 - protein, 1066 aa, Artificial Sequence
  Synthetic polypeptide
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAARDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 71 - DNA, 3204 bp, Artificial Sequence
  Synthetic polynucleotide
atgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagctttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcagcccgggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagactgagcctcaggaggagtctgaagaagaagtagaggaacctgaagaaagacagcaaacacctgaggtggtacctgatgattctggaactttctatgatcaggcagttgtcagtaatgacatggaagaacatttagaggagcctgttgctgaaccagagcctgatcctgaaccagaaccagaacaagaacctgtatctgaaatccaagaggaaaagcctgagccagtattagaagaaactgcccctgaggatgctcagaagagttcttctccagcacctgcagacatagctcagacagtacaggaagacttgaggacattttcttgggcatctgtgaccagtaagaatcttccacccagtggagctgttccagttactgggataccacctcatgttgttaaagtaccagcttcacagccccgtccagagtctaagcctgaatctcagattccaccacaaagacctcagcgggatcaaagagtgcgagaacaacgaataaatattcctccccaaaggggacccagaccaatccgtgaggctggtgagcaaggtgacattgaaccccgaagaatggtgagacaccctgacagtcaccaactcttcattggcaacctgcctcatgaagtggacaaatcagagcttaaagatttctttcaaagttatggaaacgtggtggagttgcgcattaacagtggtgggaaattacccaattttggttttgttgtgtttgatgattctgagcctgttcagaaagtccttagcaacaggcccatcatgttcagaggtgaggtccgtctgaatgtcgaagagaagaagactcgagctgccagggaaggcgaccgacgagataatcgccttcggggacctggaggccctcgaggtgggctgggtggtggaatgagaggccctccccgtggaggcatggtgcagaaaccaggatttggagtgggaagggggcttgcgccacggcagtgataa

SEQ ID NO: 72 - protein, 498 aa, Artificial sequence
  Synthetic polypeptide
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 73 - protein, 822 aa, Artificial sequence
  Synthetic polypeptide
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAATEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 74 - protein, 1066 aa, Artificial sequence
  Synthetic polypeptide
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ